(19) United States
(12) Patent Application Publication
(10) Pub. No.: US 2012/0149900 A1
(43) Pub. Date: Jun. 14, 2012

(54) FLUORINE CONTAINING COMPOUNDS AND METHODS OF USE THEREOF

(76) Inventors: Tobias Ritter, Cambridge, MA (US); Laura Brass, Newton, MA (US); Curtis Keith, Boston, MA (US); Alan Watson, Lexington, MA (US); David J. Greenblatt, Boston, MA (US)

(21) Appl. No.: 13/143,694

(22) PCT Filed: Jan. 8, 2010
(86) PCT No.: PCT/US10/20540

§ 371 Date: Feb. 27, 2012

Related U.S. Application Data
(60) Provisional application No. 61/143,667, filed on Jan. 9, 2009.
(60) Provisional application No. 61/179,331, filed on May 18, 2009.

(51) Int. Cl. C07D 489/00; C07C 69/157; C07C 225/16; C07D 413/06; C07D 221/22; C07D 223/04; C07D 233/22; C07D 233/50; C07C 211/42; C07D 211/64; C07D 489/12; C07D 211/14

(52) U.S. Cl. 544/141; 546/46; 546/45; 546/39; 560/250; 564/319; 546/237; 540/611; 548/353.1; 548/333.1; 564/427; 546/228; 546/97

(57) ABSTRACT

Fluorinated compounds and methods of making fluorinated compounds are described herein.

### RELATED APPLICATIONS

The present application claims priority under 35 U.S.C. §119(e) to U.S. provisional applications: U.S. Ser. No. 61/143,667, filed Jan. 9, 2009; and U.S. Ser. No. 61/179,331, filed May 18, 2009; each of which is incorporated herein by reference.

### BACKGROUND OF INVENTION

Functionalized fluorine containing compounds (e.g. aryl fluorides) are often used as pharmaceutical agents. In some embodiments, these products have favorable pharmacological properties such as desirable metabolic stability.

### SUMMARY OF INVENTION

Described herein are methods of making fluorine containing compounds. Also described herein are fluorinated derivatives of compounds (e.g., pharmaceutical agents). Exemplary pharmaceutical agents include a compound described herein or a fluorinated derivative thereof for use as opioid analgesics, and also compounds used to treat opioid dependence, such as an opioid analgesic or opioid dependence agent described herein.

In one aspect, the invention features a method of making a fluorinated compound, such as a compound described herein, using a method described herein.

In one aspect, the invention features a fluorinated morphine, for example, a derivative of morphine wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated morphine has the following formula:

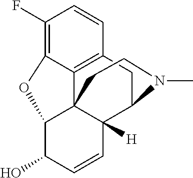

or a pharmaceutically acceptable salt thereof.

In one aspect, the invention features a method of making a fluorinated morphine, for example, the fluorinated morphine shown above, using a method described herein.

In one aspect, the invention features a fluorinated morphine-6-glucuronide, for example, a derivative of morphine-6-glucuronide wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated morphine-6-glucuronide has the following formula:

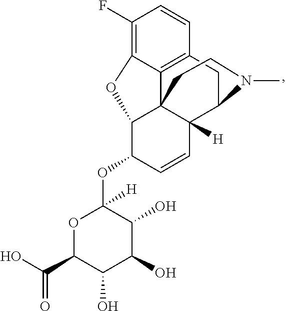

or a pharmaceutically acceptable salt thereof.

In one aspect, the invention features a method of making a fluorinated morphine-6-glucuronide, for example, the fluorinated morphine-6-glucuronide shown above, using a method described herein.

In one aspect, the invention features a fluorinated oxycodone, for example, a derivative of oxycodone wherein an aryl group has been substituted with one or more fluorine atoms, e.g., wherein a hydrogen or alkoxy substituent of an aryl group has been replaced with a fluorine. In some embodiments, the fluorine substituent is ¹⁹F. In some embodiments, the fluorine substituent is ¹⁸F. In some embodiments, the fluorinated oxycodone has the following formula:

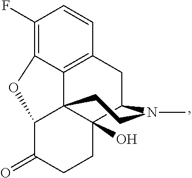

or a pharmaceutically acceptable salt thereof.

In one aspect, the invention features a method of making a fluorinated oxycodone, for example, the fluorinated oxycodone shown above, using a method described herein.

In one aspect, the invention features a fluorinated buprenorphine, for example, a derivative of buprenorphine wherein an aryl group has been substituted with one or more fluorine atoms, e.g., wherein a hydrogen or hydroxyl substituent of an aryl group has been replaced with a fluorine. In some embodiments, the fluorine substituent is ¹⁹F. In some embodiments, the fluorine substituent is ¹⁸F. In some embodiments, the fluorinated buprenorphine has the following formula:

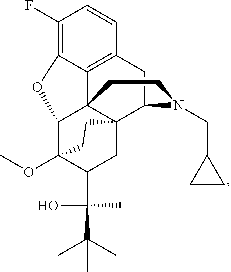

or a pharmaceutically acceptable salt thereof.

In one aspect, the invention features a method of making a fluorinated buprenorphine, for example, the fluorinated buprenorphine shown above, using a method described herein.

In one aspect, the invention features a fluorinated naloxone, for example, a derivative of naloxone wherein an aryl or heteroaryl group has been substituted with one or more fluorine atoms, e.g., wherein a hydrogen or hydroxyl substituent of an aryl or heteroaryl group has been replaced with a fluorine. In some embodiments, the fluorine substituent is ¹⁹F. In some embodiments, the fluorine substituent is ¹⁸F. In some embodiments, the fluorinated naloxone has the following formula:

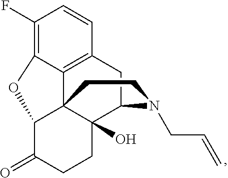

or a pharmaceutically acceptable salt thereof.

In one aspect, the invention features a method of making a fluorinated naloxone, for example, the fluorinated naloxone shown above, using a method described herein.

In one aspect, the invention features a fluorinated hydrocodone, for example, a derivative of hydrocodone wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated hydrocodone has the following formula:

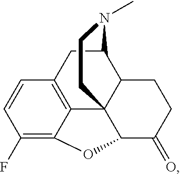

or a pharmaceutically acceptable salt thereof.

In one aspect, the invention features a method of making a fluorinated hydrocodone, for example, the fluorinated hydrocodone shown above, using a method described herein.

In one aspect, the invention features a fluorinated dextropropoxyphene, for example, a derivative of dextropropoxyphene wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated dextropropoxyphene is selected from one of the following:

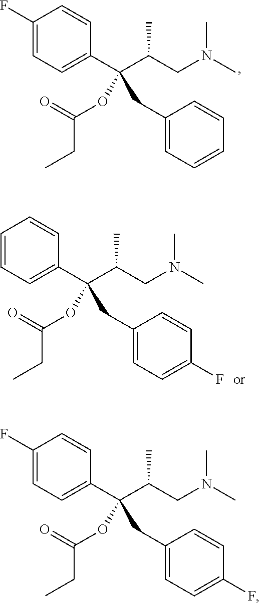

or a pharmaceutically acceptable salt thereof.

In one aspect, the invention features a method of making a fluorinated dextropropoxyphene, for example, a fluorinated dextropropoxyphene shown above, using a method described herein.

In one aspect, the invention features a fluorinated methadone, for example, a derivative of methadone wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated methadone is selected from one of the following:

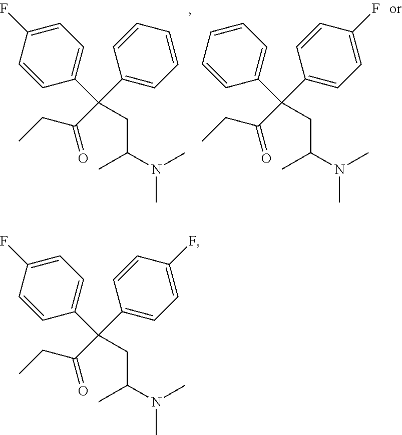

or a pharmaceutically acceptable salt thereof.

In one aspect, the invention features a method of making a fluorinated methadone, for example, a fluorinated methadone shown above, using a method described herein.

In one aspect, the invention features a fluorinated hydromorphone, for example, a derivative of hydromorphone wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated hydromorphone has the following formula:

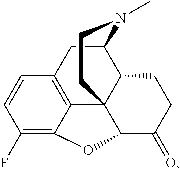

or a pharmaceutically acceptable salt thereof.

In one aspect, the invention features a method of making a fluorinated hydromorphone, for example, the fluorinated hydromorphone shown above, using a method described herein.

In one aspect, the invention features a fluorinated codeine, for example, a derivative of codeine wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated codeine has the following formula:

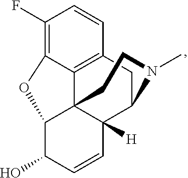

or a pharmaceutically acceptable salt thereof.

In one aspect, the invention features a method of making a fluorinated codeine, for example, the fluorinated codeine shown above, using a method described herein.

In one aspect, the invention features a fluorinated dextromoramide, for example, a derivative of dextromoramide wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated dextromoramide is selected from one of the following:

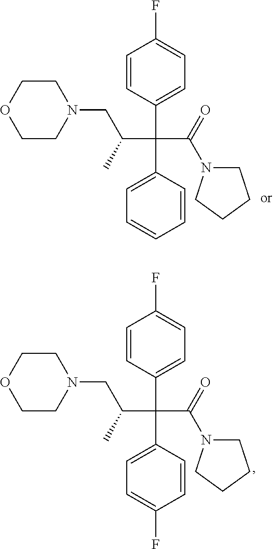

or a pharmaceutically acceptable salt thereof.

In one aspect, the invention features a method of making a fluorinated dextromoramide, for example, a fluorinated dextromoramide shown above, using a method described herein.

In one aspect, the invention features a fluorinated diamorphine (heroin), for example, a derivative of diamorphine wherein an aryl group has been substituted with one or more fluorine atoms, e.g., wherein a hydrogen or acetoxy substituent of an aryl group has been replaced with a fluorine. In some embodiments, the fluorine substituent is ¹⁹F. In some embodiments, the fluorine substituent is ¹⁸F. In one embodiment, the fluorinated diamorphine has the following formula:

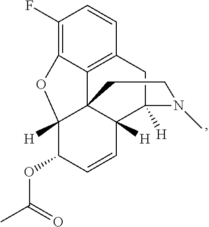

or a pharmaceutically acceptable salt thereof.

In one aspect, the invention features a method of making a fluorinated diamorphine, for example, the fluorinated diamorphine shown above, using a method described herein.

In one aspect, the invention features a fluorinated dihydrocodeine, for example, a derivative of dihydrocodeine wherein an aryl group has been substituted with one or more fluorine atoms, e.g., wherein a hydrogen or methoxy substituent of an aryl group has been replaced with a fluorine. In some embodiments, the fluorine substituent is ¹⁹F. In some embodiments, the fluorine substituent is ¹⁸F. In one embodiment, the fluorinated dihydrocodeine has the following formula:

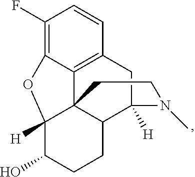

or a pharmaceutically acceptable salt thereof.

In one aspect, the invention features a method of making a fluorinated dihydrocodeine, for example, the fluorinated dihydrocodeine shown above, using a method described herein.

In one aspect, the invention features a fluorinated dipipanone, for example, a derivative of dipipanone wherein an aryl group has been substituted with one or more fluorine atoms, e.g., wherein a hydrogen of an aryl group has been replaced with a fluorine. In some embodiments, the fluorine substituent is ¹⁹F. In some embodiments, the fluorine substituent is ¹⁸F. In one embodiment, the fluorinated dipipanone is selected from one of the following:

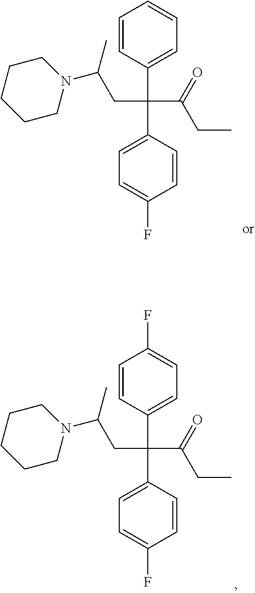

or a pharmaceutically acceptable salt thereof.

In one aspect, the invention features a method of making a fluorinated dipipanone, for example, the fluorinated dipipanone shown above, using a method described herein.

In one aspect, the invention features a fluorinated meptazinol, for example, a derivative of meptazinol wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated meptazinol is selected from one of the following:

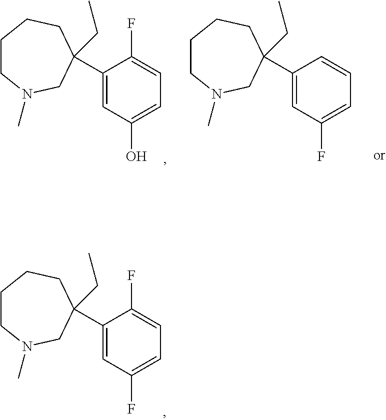

or a pharmaceutically acceptable salt thereof.

In one aspect, the invention features a method of making a fluorinated meptazinol, for example, the fluorinated meptazinol shown above, using a method described herein.

In one aspect, the invention features a fluorinated nalbuphine, for example, a derivative of nalbuphine wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated nalbuphine has the following formula:

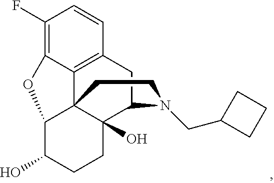

or a pharmaceutically acceptable salt thereof.

In one aspect, the invention features a method of making a fluorinated nalbuphine, for example, the fluorinated nalbuphine shown above, using a method described herein.

In one aspect, the invention features a fluorinated lofexidine, for example, a derivative of lofexidine wherein an aryl group has been substituted with one or more fluorine atoms, e.g., wherein a hydrogen or halogen substituent of an aryl group has been replaced with a fluorine. In some embodiments, the fluorine substituent is ¹⁹F. In some embodiments, the fluorine substituent is ¹⁸F. In one embodiment, the fluorinated lofexidine is selected from one of the following:

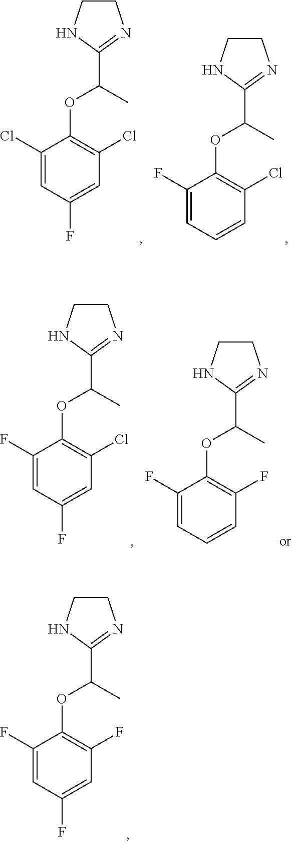

or a pharmaceutically acceptable salt thereof.

In one aspect, the invention features a method of making a fluorinated lofexidine, for example, a fluorinated lofexidine shown above, using a method described herein.

In one aspect, the invention features a fluorinated naltrexone, for example, a derivative of naltrexone wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated naltrexone has the following formula:

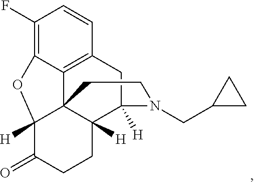

or a pharmaceutically acceptable salt thereof.

In one aspect, the invention features a method of making a fluorinated naltrexone, for example, the fluorinated naltrexone shown above, using a method described herein.

In one aspect, the invention features a fluorinated oxymorphone, for example, a derivative of oxymorphone wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In some embodiments, the fluorinated oxymorphone has the following formula:

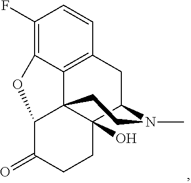

or a pharmaceutically acceptable salt thereof.

In one aspect, the invention features a method of making a fluorinated oxymorphone, for example, the fluorinated oxymorphone shown above, using a method described herein.

In one aspect, the invention features a fluorinated nalorphine, for example, a derivative of nalorphine wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated nalorphine has the following formula:

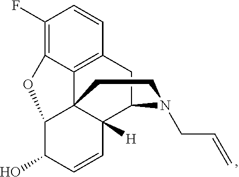

or a pharmaceutically acceptable salt thereof.

In one aspect, the invention features a method of making a fluorinated nalorphine, for example, the fluorinated nalorphine shown above, using a method described herein.

In one aspect, the invention features a fluorinated etorphine, for example, a derivative of etorphine wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated etorphine has the following formula:

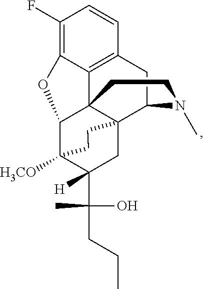

or a pharmaceutically acceptable salt thereof.

In one aspect, the invention features a method of making a fluorinated etorphine, for example, the fluorinated etorphine shown above, using a method described herein.

In one aspect, the invention features a fluorinated dihydroetorphine, for example, a derivative of dihydroetorphine wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated dihydroetorphine has the following formula:

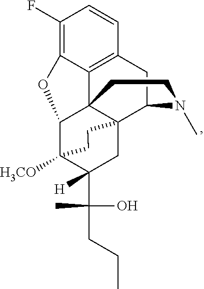

or a pharmaceutically acceptable salt thereof.

In one aspect, the invention features a method of making a fluorinated dihydroetorphine, for example, the fluorinated dihydroetorphine shown above, using a method described herein.

In one aspect, the invention features a fluorinated N-phenethyl-14-ethoxymetopon, for example, a derivative of N-phenethyl-14-ethoxymetopon wherein an aryl group has been substituted with one or more fluorine atoms, e.g., wherein a hydrogen or hydroxy substituent of an aryl group has been replaced with a fluorine. In some embodiments, the fluorine substituent is ¹⁹F. In some embodiments, the fluorine substituent is ¹⁸F. In one embodiment, the fluorinated N-phenethyl-14-ethoxymetopon has the following formula:

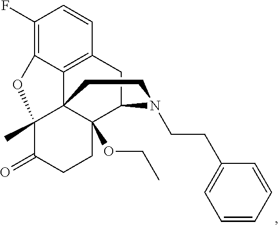

or a pharmaceutically acceptable salt thereof.

In one aspect, the invention features a method of making a fluorinated N-phenethyl-14-ethoxymetopon, for example, the fluorinated N-phenethyl-14-ethoxymetopon shown above, using a method described herein.

In one aspect, the invention features a fluorinated thebaine, for example, a derivative of thebaine wherein an aryl group has been substituted with one or more fluorine atoms, e.g., wherein a hydrogen or alkoxy substituent of an aryl group has been replaced with a fluorine. In some embodiments, the fluorine substituent is ¹⁹F. In some embodiments, the fluorine substituent is ¹⁸F. In one embodiment, the fluorinated thebaine has the following formula:

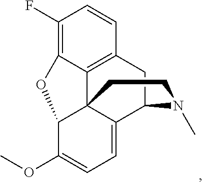

or a pharmaceutically acceptable salt thereof.

In one aspect, the invention features a method of making a fluorinated thebaine, for example, the fluorinated thebaine shown above, using a method described herein.

In one aspect, the invention features a method of making a fluorinated fentanyl, for example, a fluorinated fentanyl with the following formula, using a method described herein:

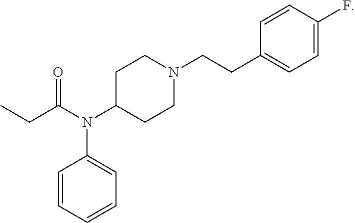

In one aspect, the invention features an ¹⁸F-substituted clonidine, for example, a derivative of clonidine wherein an aryl group has been substituted with one or more ¹⁸F atoms, e.g., wherein a hydrogen or halogen substituent of an aryl group has been replaced with ¹⁸F. In one embodiment, the ¹⁸F-substituted clonidine is selected from one of the following:

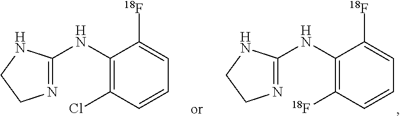

or a pharmaceutically acceptable salt thereof.

In one aspect, the invention features a method of making a fluorinated clonidine, for example, a fluorinated clonidine with one of the following formulae, using a method described herein:

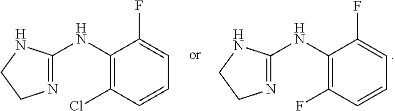

In one aspect, the invention features an ¹⁸F-substituted pentazocine, for example, a derivative of pentazocine wherein an aryl group has been substituted with one or more ¹⁸F atoms, e.g., wherein a hydrogen or hydroxy substituent of an aryl group has been replaced with ¹⁸F. In one embodiment, the ¹⁸F-substituted pentazocine has the following formula:

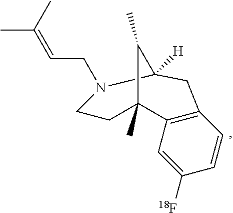

or a pharmaceutically acceptable salt thereof.

In one aspect, the invention features a method of making a fluorinated pentazocine, for example, a fluorinated pentazocine with the following formula, using a method described herein:

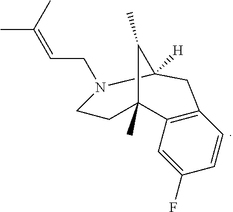

In one aspect, the invention features an ¹⁸F-substituted pethidine, for example, a derivative of pethidine wherein an aryl group has been substituted with one or more ¹⁸F atoms, e.g., wherein a hydrogen of an aryl group has been replaced with ¹⁸F. In one embodiment, the ¹⁸F-substituted pethidine has the following formula:

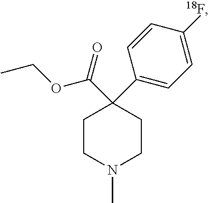

or a pharmaceutically acceptable salt thereof.

In one aspect, the invention features a method of making a fluorinated pethidine, for example, a fluorinated pethidine with the following formula, using a method described herein:

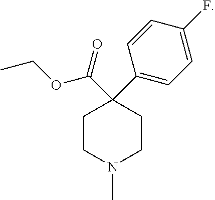

In one aspect, the invention features a fluorinated phenazocine, for example, a derivative of phenazocine wherein an aryl or heteroaryl group has been substituted with one or more fluorine atoms, e.g., wherein a hydrogen or halogen substituent of an aryl or heteroaryl group has been replaced with a fluorine. In some embodiments, the fluorinated phenazocine does not have the following formula:

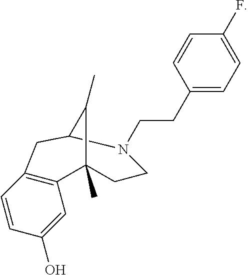

In some embodiments, the fluorine substituent is ¹⁹F. In some embodiments, the fluorine substituent is ¹⁸F. In some embodiments, the fluorinated phenazocine is selected from one of the following:

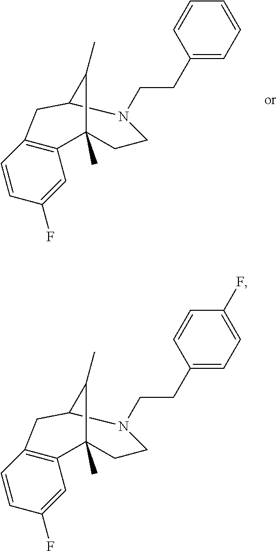

or a pharmaceutically acceptable salt thereof.

In one aspect, the invention features a method of making a fluorinated phenazocine, including any of the three fluorinated phenazocine structures shown above, using a method described herein.

In one aspect, the invention features an ¹⁸F-substituted phenazocine, for example, a derivative of phenazocine wherein an aryl or heteroaryl group has been substituted with one or more ¹⁸F atoms, e.g., wherein a hydrogen or halogen substituent of an aryl or heteroaryl group has been replaced with ¹⁸F. In one embodiment, the ¹⁸F-substituted phenazocine has the following formula:

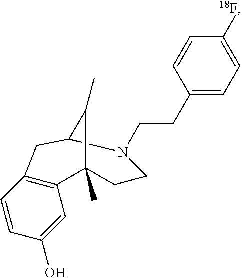

or a pharmaceutically acceptable salt thereof.

In one aspect, the invention features a composition comprising a compound described herein (e.g., a pharmaceutical composition comprising a compound described herein).

In one aspect, the invention features a kit comprising a compound or composition described herein.

In some embodiments, a compound described herein can be administered to a subject to treat a disorder described herein, e.g., a disorder that can be treated with an opioid analgesic, or an opioid dependence disorder.

In some embodiments, a compound described herein (e.g., a fluorinated derivative of a pharmaceutical agent) has one or more properties that are superior to a corresponding unfluorinated derivatives of that pharmaceutical agent (e.g., where the corresponding unfluorinated derivative is either without a fluorine in the structure or does not include the same fluorine substitution pattern as the fluorinated derivative described herein). In some embodiments, the improved property is improved metabolic stability, improved penetration across the blood brain barrier, reduced penetration across the blood brain barrier, or improved solubility.

The term “halo” or “halogen” refers to any radical of fluorine, chlorine, bromine or iodine.

The term “alkyl” refers to a hydrocarbon chain that may be a straight chain or branched chain, containing the indicated number of carbon atoms. For example, C₁-C₁₂ alkyl indicates that the group may have from 1 to 12 (inclusive) carbon atoms in it. The term “haloalkyl” refers to an alkyl in which one or more hydrogen atoms are replaced by halo, and includes alkyl moieties in which all hydrogens have been replaced by halo (e.g., perfluoroalkyl).

The term, “cyano” refers to a —CN radical.

The terms “alkylamino” and “dialkylamino” refer to —NH(alkyl) and —NH(alkyl)₂ radicals respectively. The term “hydroxy” refers to an OH radical. The term “alkoxy” refers to an —O-alkyl radical. The term “mercapto” refers to an SH radical. The term “thioalkoxy” refers to an —S-alkyl radical.

The term “aryl” refers to an aromatic monocyclic, bicyclic, or tricyclic hydrocarbon ring system, wherein any ring atom capable of substitution can be substituted (e.g., by one or more substituents). Examples of aryl moieties include, but are not limited to, phenyl, naphthyl, and anthracenyl.

The term “cycloalkyl” as employed herein includes saturated cyclic, bicyclic, tricyclic, or polycyclic hydrocarbon groups having 3 to 12 carbons. Any ring atom can be substituted (e.g., by one or more substituents). The cycloalkyl groups can contain fused rings. Fused rings are rings that share a common carbon atom. Examples of cycloalkyl moieties include, but are not limited to, cyclopropyl, cyclohexyl, methylcyclohexyl, adamantyl, and norbornyl.

The term “heterocyclyl” refers to a nonaromatic 3-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The heteroatom may optionally be the point of attachment of the heterocyclyl substituent. Any ring atom can be substituted (e.g., by one or more substituents). The heterocyclyl groups can contain fused rings. Fused rings are rings that share a common carbon atom. Examples of heterocyclyl include, but are not limited to, tetrahydrofuranyl, tetrahydropyranyl, piperidinyl, morpholino, pyrrolinyl, pyrimidinyl, quinolinyl, and pyrrolidinyl.

The term “cycloalkenyl” refers to partially unsaturated, nonaromatic, cyclic, bicyclic, tricyclic, or polycyclic hydrocarbon groups having 5 to 12 carbons, preferably 5 to 8 carbons. The unsaturated carbon may optionally be the point of attachment of the cycloalkenyl substituent. Any ring atom can be substituted (e.g., by one or more substituents). The cycloalkenyl groups can contain fused rings. Fused rings are rings that share a common carbon atom. Examples of cycloalkenyl moieties include, but are not limited to, cyclohexenyl, cyclohexadienyl, or norbornenyl.

The term “heterocycloalkenyl” refers to a partially saturated, nonaromatic 5-10 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). The unsaturated carbon or the heteroatom may optionally be the point of attachment of the heterocycloalkenyl substituent. Any ring atom can be substituted (e.g., by one or more substituents). The heterocycloalkenyl groups can contain fused rings. Fused rings are rings that share a common carbon atom. Examples of heterocycloalkenyl include but are not limited to tetrahydropyridyl and dihydropyranyl.

The term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Any ring atom can be substituted (e.g., by one or more substituents).

The term “acyl” refers to an alkylcarbonyl, cycloalkylcarbonyl, arylcarbonyl, heterocyclylcarbonyl, or heteroarylcarbonyl substituent, any of which may be further substituted (e.g., by one or more substituents).

### BRIEF DESCRIPTION OF DRAWINGS

FIG. 1 is a depiction of a competition curve obtained with fluorinated morphine at the non-selective opioid receptor.

FIG. 2 is a depiction of a competition curve obtained with fluorinated morphine at the kappa (KOP) receptor.

FIG. 3 is a depiction of a competition curve obtained with fluorinated morphine at the human mu (MOP) receptor.

FIG. 4 is a depiction of a competition curve obtained with fluorinated morphine at the human delta 2 (DOP) receptor.

FIG. 5 is a depiction of plasma and brain distribution of fluorinated morphine or morphine following single 1 mg/kg iv dose administered in rats.

FIG. 6 is a depiction of pharmacokinetic parameters following single 1 mg/kg iv dose of fluorinated morphine administered in rats.

FIG. 7 is a depiction of a proposed PK model for IV administration of fluorinated morphine.

### DETAILED DESCRIPTION

Compounds

Described herein are fluorinated compounds, e.g., fluorinated derivatives of a pharmaceutical agent. In some embodiments, the compound includes one or more fluorine moieties on an aryl or heteroaryl ring within the pharmaceutical agent.

In some embodiments the compound is a fluorinated derivative of an opioid analgesic or an opioid dependence agent (e.g., an opioid receptor agonist or an opioid receptor antagonist). Exemplary compounds include the following:

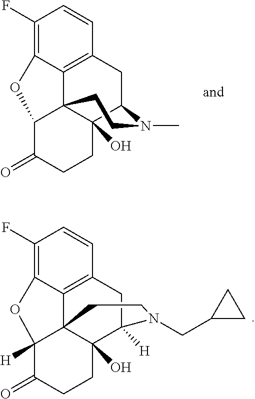

The compounds of this invention may contain one or more asymmetric centers and thus occur as racemates and racemic mixtures, single enantiomers, individual diastereomers and diastereomeric mixtures. All such isomeric forms of these compounds are expressly included in the present invention. The compounds of this invention may also contain linkages (e.g., carbon-carbon bonds) or substituents that can restrict bond rotation, e.g. restriction resulting from the presence of a ring or double bond. Accordingly, all cis/trans and E/Z isomers are expressly included in the present invention.

The compounds of this invention may also be represented in multiple tautomeric forms. In such instances, the invention expressly includes all tautomeric forms of the compounds described herein, even though only a single tautomeric form may be represented (e.g., alkylation of a ring system may result in alkylation at multiple sites, the invention expressly includes all such reaction products). All such isomeric forms of such compounds are expressly included in the present invention. All crystal forms of the compounds described herein are expressly included in the present invention.

The compounds of this invention include the compounds themselves, as well as their salts and their prodrugs, if applicable. A salt, for example, can be formed between an anion and a positively charged substituent (e.g., amino) on a compound described herein. Suitable anions include chloride, bromide, iodide, sulfate, nitrate, phosphate, citrate, methanesulfonate, trifluoroacetate, and acetate. Likewise, a salt can also be formed between a cation and a negatively charged substituent (e.g., carboxylate) on a compound described herein. Suitable cations include sodium ion, potassium ion, magnesium ion, calcium ion, and an ammonium cation such as tetramethylammonium ion. Examples of prodrugs include esters and other pharmaceutically acceptable derivatives, which, upon administration to a subject, are capable of providing active compounds.

The compounds of this invention may be modified by appending appropriate functionalities to enhance selected biological properties, e.g., targeting to a particular tissue. Such modifications are known in the art and include those which increase biological penetration into a given biological compartment (e.g., blood, lymphatic system, central nervous system), increase oral availability, increase solubility to allow administration by injection, alter metabolism and alter rate of excretion.

In an alternate embodiment, the compounds described herein may be used as platforms or scaffolds that may be utilized in combinatorial chemistry techniques for preparation of derivatives and/or chemical libraries of compounds. Such derivatives and libraries of compounds have biological activity and are useful for identifying and designing compounds possessing a particular activity. Combinatorial techniques suitable for utilizing the compounds described herein are known in the art as exemplified by Obrecht, D. and Villalgrodo, J. M., Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, A. W., Curr. Opin. Chem. Bio., (1997) 1, 60). Thus, one embodiment relates to a method of using the compounds described herein for generating derivatives or chemical libraries comprising: 1) providing a body comprising a plurality of wells; 2) providing one or more compounds identified by methods described herein in each well; 3) providing an additional one or more chemicals in each well; 4) isolating the resulting one or more products from each well. An alternate embodiment relates to a method of using the compounds described herein for generating derivatives or chemical libraries comprising: 1) providing one or more compounds described herein attached to a solid support; 2) treating the one or more compounds identified by methods described herein attached to a solid support with one or more additional chemicals; 3) isolating the resulting one or more products from the solid support. In the methods described above, “tags” or identifier or labeling moieties may be attached to and/or detached from the compounds described herein or their derivatives, to facilitate tracking, identification or isolation of the desired products or their intermediates. Such moieties are known in the art. The chemicals used in the aforementioned methods may include, for example, solvents, reagents, catalysts, protecting group and deprotecting group reagents and the like. Examples of such chemicals are those that appear in the various synthetic and protecting group chemistry texts and treatises referenced herein.

Synthetic Methods

Described herein are methods of making a fluorine-containing compound (e.g., a compound described herein). The compounds described herein can be synthesized via a variety of methods, included Ag or Pd mediated methods. In general, the methods include an organic compound to be fluorinated, a fluorinating agent, and either a silver salt or a palladium complex.

Compounds to be Fluorinated

Exemplary compounds such as a pharmaceutical agent or a precursor thereof or a derivative thereof, include those described herein. The compound may be a small organic molecule or a large organic molecule. A small organic molecule includes any molecule having a molecular weight of less than 1000 g/mol, of less than 900 g/mol, of less than 800 g/mol, of less than 700 g/mol, of less than 600 g/mol, of less than 500 g/mol, of less than 400 g/mol, of less than 300 g/mol, of less than 200 g/mol or of less than 100 g/mol. A large organic molecule include any molecule of between 1000 g/mol to 5000 g/mol, of between 1000 g/mol to 4000 g/mol, of between 1000 g/mol to 3000 g/mol, of between 1000 g/mol to 2000 g/mol, or of between 1000 g/mol to 1500 g/mol. Organic compounds include aryl compounds, heteroaryl compounds, carbocyclic compounds, heterocyclic compounds, aliphatic compounds, heteroaliphatic compounds. In a preferred embodiment, the organic compound is an aryl compound (e.g., a phenyl compound), or a heteroaryl compound (e.g. a quinolyl or indolyl compound).

In some embodiments, the compound contains a chiral center. In some embodiments, the compound is further substituted with one or more functional groups (e.g., alcohols, aldehydes, ketones, alkenes, alkoxy groups, cyano groups, amides and N-oxides). In some embodiments, the functional groups are unprotected. In some embodiments, the compound is a precursor of a pharmaceutically acceptable compound.

Fluorinating Agents

As generally described above, the process utilizes a fluorinating agent. In some embodiments, the fluorinating agent is an electrophilic fluorinating agent. In some embodiments, the fluorinating agent is commercially available. In some embodiments, the electrophilic fluorinating agent is also an inorganic fluorinating agent. Exemplary electrophilic fluorinating agents include, but are not limited to, N-fluoropyridinium triflate, N-fluoro-2,4,6-trimethylpyridinium triflate, N-fluoro-2,4,6-trimethylpyridinium tetrafluoroborate, N-fluoro-2,6-dichloropyridinium tetrafluoroborate, N-fluoro-2,6-dichloropyridinium triflate, N-fluoropyridinium pyridine heptafluorodiborate, N-fluoropyridinium tetrafluoroborate, N-fluoropyridinium triflate, an N-fluoroarylsulfonimide (e.g., N-fluorobenzenesulfonimide), N-chloromethyl-N′-fluorotriethylenediammonium bis(tetrafluoroborate) (Selectfluor®), N-chloromethyl-N′-fluorotriethylenediammonium bis(hexafluorophosphate), N-chloromethyl-N′-fluorotriethylenediammonium bis(triflate) and XeF₂. In some embodiments, the fluorinating agent is Selectfluor®. In some embodiments, the fluorinating agent is N-fluoropyridinium triflate. In some embodiments, the fluorinating agent is N-fluoro-2,4,6-trimethylpyridinium triflate. In some embodiments, the fluorinating agent is N-fluoro-2,4,6-trimethylpyridinium tetrafluoroborate. In some embodiments, the fluorinating agent is N-fluoro-benzenesulfonimide. In some embodiments, the fluorinating agent is xenon difluoride.

The fluorinating agent may be enriched with a particular isotope of fluorine. In some embodiments, the fluorinating agent is labeled with ¹⁹F (i.e., transfers an ¹⁹F fluorine substituent to the organic compound). In some embodiments, reaction of the ¹⁹F fluorinating agent in the process provides a fluorinated ¹⁹F-labeled organic compound.

In some embodiments, the fluorinating agent is labeled with ¹⁸F (i.e., transfers an ¹⁸F fluorine substituent to the organic compound). In some embodiments, reaction of the ¹⁸F fluorinating agent in the process provides a fluorinated ¹⁸F-labeled organic compound.

However, in some embodiments, the fluorinating agent is labeled with a mixture of ¹⁸F and ¹⁹F. In some embodiments, reaction of the mixture of ¹⁹F and ¹⁸F fluorinating agent in the process provides a mixture of fluorinated ¹⁹F-labeled organic compound and fluorinated ¹⁸F-labeled organic compound.

Any of the above fluorinated agents may be labeled as ¹⁹F or ¹⁸F.

For example, in some embodiments, the fluorinating agent is ¹⁹F-labeled N-(chloromethyl)-N′-fluorotriethylenediamine bis(tetrafluoroborate) (Selectfluor®) or ¹⁹F-labeled XeF₂. In some embodiments, the fluorinating agent is ¹⁹F-labeled N-(chloromethyl)-N′-fluorotriethylenediamine bis(tetrafluoroborate) (Selectfluor®). In some embodiments, the fluorinating agent is ¹⁹F-labeled XeF₂.

In some embodiments, the fluorinating agent is ¹⁸F-labeled N-(chloromethyl)-N′-fluorotriethylenediamine bis(tetrafluoroborate) (Selectfluor®) or ¹⁸F-labeled XeF₂. In some embodiments, the fluorinating agent is ¹⁸F-labeled N-(chloromethyl)-N′-fluorotriethylenediamine bis(tetrafluoroborate) (Selectfluor®). In some embodiments, the fluorinating agent is ¹⁸F-labeled XeF₂.

Exemplary methods include the following.

### Ag(I)-Mediated Fluorination

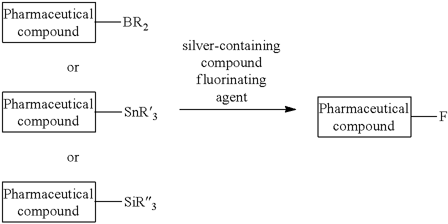

Upon reaction of an organic compound comprising an organostannane, a boron substituent or a silane substituent, with a silver-containing compound and a fluorinating agent, the method provides a fluorinated organic compound in which the organostannane, boron substituent or silane substituent is replaced with a fluorine substituent. In some embodiments, the organostannane, boron substituent or silane substituent is attached to an aryl or heteroaryl moiety of the organic compound. For examples, see Schemes 1-6.

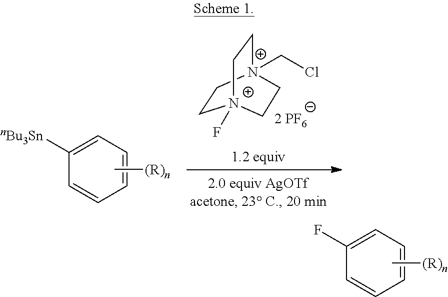

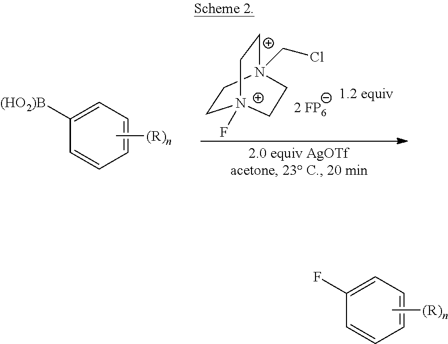

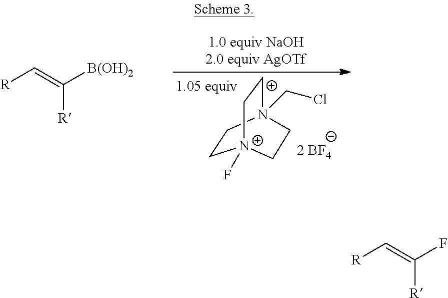

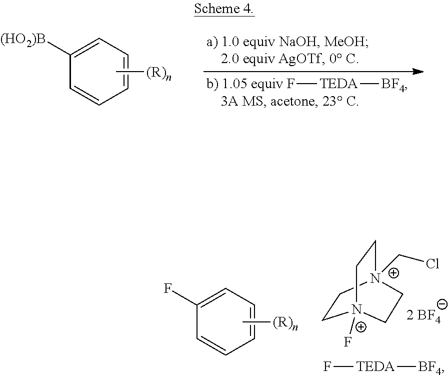

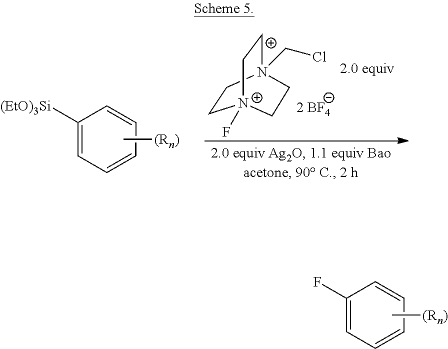

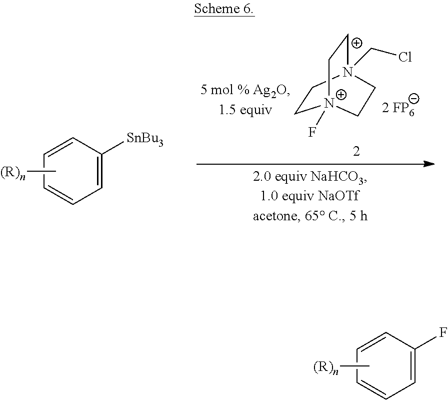

In the above Schemes 1-6, R and R′ are substituents and n may be 0, 1, 2, 3, 4 or 5. Exemplary substituents include, without limitation, alkyl (e.g., C1, C2, C3, C4, C5, C6, C7, C8, C9, C10, C11, C12 straight or branched chain alkyl), cycloalkyl, haloalkyl (e.g., perfluoroalkyl such as CF₃), aryl, heteroaryl, aralkyl, heteroaralkyl, heterocyclyl, alkenyl, alkynyl, cycloalkenyl, heterocycloalkenyl, alkoxy, haloalkoxy (e.g., perfluoroalkoxy such as OCF₃), halo, hydroxy, carboxy, carboxylate, cyano, nitro, amino, alkylamino, dialkylamino, SO₃H, sulfate, phosphate, methylenedioxy (—O—CH₂—O— wherein oxygens are attached to vicinal atoms), ethylenedioxy, oxo, thioxo (e.g., C═S), imino (alkyl, aryl, aralkyl), S(O)ₙalkyl (where n is 0-2), S(O)ₙ aryl (where n is 0-2), S(O)ₙ heteroaryl (where n is 0-2), S(O)ₙ heterocyclyl (where n is 0-2), amine (mono-, di-, alkyl, cycloalkyl, aralkyl, heteroaralkyl, aryl, heteroaryl, and combinations thereof), ester (alkyl, aralkyl, heteroaralkyl, aryl, heteroaryl), amide (mono-, di-, alkyl, aralkyl, heteroaralkyl, aryl, heteroaryl, and combinations thereof), sulfonamide (mono-, di-, alkyl, aralkyl, heteroaralkyl, and combinations thereof). The substituents are independently any one single, or any subset of the aforementioned substituents. A substituent may itself be substituted with any one of the above substituents. In some embodiments, two R groups may be taken together to form a ring, e.g., an aryl, heteroaryl, cyclyl or heterocyclyl ring, which may itself be further substituted with any one of the above substituents.

In some embodiments, the method uses a catalytic amount of silver. Exemplary methods of fluorinating a compound using Ag are described in P.C.T. Application No. PCT/US2009/065339, filed Nov. 20, 2009, which is incorporated herein by reference in its entirety.

Boron Substituents

Methods of fluorinating an organic compound are described herein. In some embodiments, the organic compound comprises a boron substituent. The boron substituent may be of the formula:

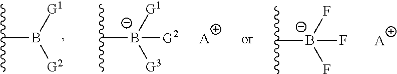

wherein G¹, G² and G³ are, independently, —OH, —OR, or —R, wherein each R is, independently, optionally substituted aliphatic, optionally substituted heteroaliphatic, optionally substituted aryl, or optionally substituted heteroaryl, or G¹ and G² are joined to form an optionally substituted 5- to 8-membered ring having at least one O atom directly attached to B, wherein the ring is comprised of carbon atoms and optionally one or more additional heteroatoms independently selected from the group consisting of N, S, and O. A⁺ may be a metal cation or ammonium.

As used herein, a boron substituent is intended to encompass free boronic acid substituents (i.e., wherein G¹ and G² are both —OH) and oligomeric anhydrides thereof (including dimers, trimers, and tetramers, and mixtures thereof), boronic ester substituents (i.e., wherein G¹ is —OH or —OR and G² is —OR), borinic acid substituents (i.e., wherein G¹ is —OH and G² is —R), borinic ester substituents (i.e., wherein G¹ is —OR and G² is —R), trihydroxoborates (i.e., wherein G¹, G² and G³ are all —OH), and trialkoxyborates (i.e., wherein G¹, G² and G³ are all —OR, e.g., —OCH₃).

In some embodiments, G¹ and G² are joined to form a 5-membered ring. Exemplary 5-membered rings include:

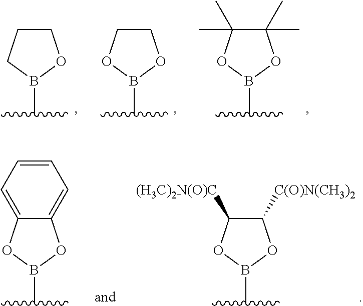

In some embodiments, G¹ and G² are joined to form a 6-membered ring. Exemplary 6-membered rings include:

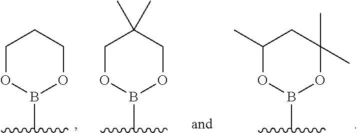

In some embodiments, G¹ and G² are joined to form an 8-membered ring. Exemplary 8-membered rings include:

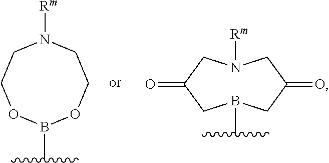

wherein R^{m} is hydrogen, a suitable amino protecting group, or an optionally substituted aliphatic, optionally substituted heteroaliphatic, optionally substituted aryl, or optionally substituted heteroaryl group.

Furthermore, as used herein, a boron substituent is also intended to encompass a trifluoroborate substituent. For example, in some embodiments, a boron substituent is a group of the formula:

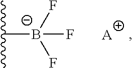

wherein A^{⊕} is a metal cation or ammonium.

Furthermore, as used herein, a boron substituent is also intended to encompass trihydroxy- and trialkoxy borates. For example, in some embodiments, a boron substituent is a group of the formulae:

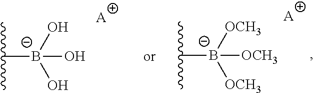

wherein A^{⊕} is a metal cation or ammonium.

Exemplary metal cations include lithium, sodium, potassium, magnesium, and calcium cations. In some embodiments, the metal cation is a potassium cation.

An organic compound comprising a boron substituent may be obtained via a variety of known methods. For example, a halogen-containing precursor may be reacted with a boron-containing compound to generate the organic compound comprising a boron substituent. An unactivated C—H bond may also be borylated, for example, using a suitable catalyst.

Silane Substituents

Methods of fluorinating an organic compound are described herein. In some embodiments, the organic compound comprises a silane substituent. The silane substituent may be a trialkoxysilane, e.g., trimethoxysilane or triethoxysilane. The silane substituent may be a trihydroxysilane.

An organic compound comprising a silane substituent may be obtained via a variety of known methods. For example, a Grignard-containing precursor may be reacted with a silicon-containing compound (e.g., a tetraalkoxysilane) to generate the organic compound comprising a silane substituent. In another example, a halogen-containing precursor or a triflyl-containing precursor may be reacted with a silicon-containing compound (e.g., a tetraalkoxysilane) in the presence of a suitable catalyst (e.g., a Pd⁰ or Rh^{I} catalyst) to generate the organic compound comprising a silane substituent.

Organostannanes

Methods of fluorinating an organic compound are described herein. In some embodiments, the organic compound comprises an organostannane. The organostannane may be a trialkylstannane, e.g., trimethylstannane or tributylstannane.

Silver-Containing Compounds

The Ag methods described herein generally include a silver-containing compound. The silver-containing compound may be a silver complex or a silver salt, e.g., a silver(I) salt. Exemplary silver salts include silver(I) salts such as silver(I) fluoride, silver(I) acetate, silver(I) tetrafluoroborate, silver(I) perchlorate, silver(I) nitrate, silver(I) carbonate, silver(I) cyanide, silver(I) benzoate, silver(I) triflate, silver(I) hexafluorophosphate, silver(I) hexafluoroantimonate, silver(I) oxide, silver(I) nitrite and silver(I) phosphate. In preferred embodiments, the silver salt is silver(I) triflate or silver(I) oxide.

### Pd(II)-Mediated Fluorination

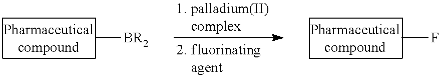

Upon reaction of an organic compound comprising a boron substituent with a palladium(II) complex and a fluorinating agent, the method provides a fluorinated organic compound in which the boron substituent is replaced with a fluorine substituent. In some embodiments, the boron substituent is attached to an aryl or heteroaryl moiety of the organic compound. For example, see Scheme 7.

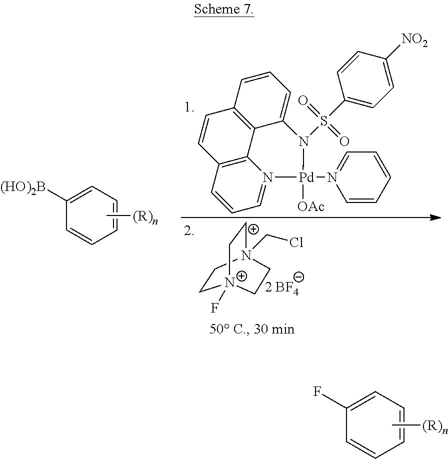

Exemplary methods of fluorinating a compound using a Pd(II) complex are described in WO2009/100014, which is incorporated herein by reference in its entirety.
Palladium (II) complexes

In some embodiments, a stoichiometric amount of the palladium (II) complex is used.

In some embodiments, the palladium (II) complex comprises a bidentate ligand. In some embodiments, the palladium (II) complex comprises a tridentate ligand.

In some embodiments, the palladium (II) complex is crystalline. Alternatively, in some embodiments, the palladium (II) complex is amorphous.

In some embodiments, the palladium (II) complex is not a salt. Alternatively, in some embodiments, the palladium (II) complex is a salt. For example, in some embodiments, the palladium (II) complex is a salt of tetrafluoroborate (BF₄⁻), tetraphenylborate (BPh₄⁻), phosphorous hexafluoride (PF6⁻), BArF⁻ tetrakis(pentafluorophenyl)borate, antimohexafluoride (SbF₆⁻), or trifluoromethansulfonate (triflate, CF₃SO₃⁻). In some embodiments, the palladium (II) complex is a salt of tetrafluoroborate (BF₄⁻).

In some embodiments, the palladium (II) complex is a palladium (II) dimer complex.

In some embodiments, the palladium (II) complex is generated in situ from a complex in the 0 oxidation state (i.e., a “palladium (0) complex”) and one or more ligands.

Exemplary ligands include, but are not limited to, halogens (e.g., iodide, bromide, chloride, fluoride), solvents (e.g., hydroxide, water, ammonia, acetonitrile, dimethylsulfoxide, dimethylformamide, dimethylacetamide), sulfide, cyanide, carbon monoxide, thiocyanate, isothiocyanate, nitrate, nitrite, azide, oxalate, olefins (e.g., dibenzylidineacetone (dba)), optionally substituted pyridines (py) (e.g., 2,2′,5′,2-terpyridine (terpy), bipyridine (bipy) and other pyridine ligands as described herein), optionally substituted aryl (e.g., phenyl (Ph), phenanthroline (phen), biphenyl), phosphines (e.g., triphenylphosphine (PPh₃), 1,2-bis(diphenylphosphino)ethane (dppe), tricyclohexylphosphine (PCy₃), tri(o-tolyl)phosphine (P(o-tol)₃), tris(2-diphenylphosphineethyl)amine (np3)), amino ligands (e.g., ethylenediamine (en), diethylenetriamine (dien), tris(2-aminoethyl)amine (tren), triethylenetetramine (trien), ethylenediaminetetraacetate (EDTA)), acyloxy ligands (e.g., acetylacetonate (acac), O-acetate (—OAc)), and alkyloxy ligands (e.g., —OMe, OiPr, OtBu).

As one of ordinary skill in the art would understand, the ligands are chosen to satisfy the valency of palladium. Thus, in some embodiments, the ligands are chosen to satisfy the valency of a palladium complex as +2.

Exemplary palladium (II) complexes include, but are not limited to, palladium (II) bromide, palladium (II) chloride, palladium (II) iodide, palladium (II) fluoride, palladium (II) acetate, palladium (II) acetylacetonate, palladium (II) oxide, palladium (II) cyanide, palladium (II) sulfide, palladium (II) sulfate, palladium (II) 2,4-pentanedionate, allyl palladium (II) chloride dimer, bis(acetonitrile)dichloropalladium (II), trans-bis(benzonitrile)dichloropalladium (II), and trichloro-bis(triphenylphosphine)palladium (II).

Exemplary palladium (0) complexes include, but are not limited to, Pd₂ dba₃, Pd₂ dba₃-CHCl₃, and tetrakis(triphenylphosphine)palladium (0).

Other exemplary ligands are provided as groups R^{L1} and R^{L2}, described below and herein. Furthermore, other exemplary bidentate and tridentate palladium (II) complexes are provided in the following formulae, described below and herein.

For example, in some embodiments, the palladium (II) complex comprises a bidentate or tridentate ligand to provide a complex of the formula (I):

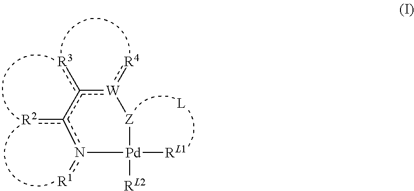

wherein:

Pd represents palladium of valency of +2;

R^{L1} and R^{L2} are, independently, optionally substituted aliphatic, optionally substituted heteroaliphatic, optionally substituted aryl, optionally substituted heteroaryl, halogen, —OR^{a}, —SR^{b}, —N(R^{c})₂, —N(R^{c})₃, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, optionally substituted heteroaliphatic, optionally substituted aryl, optionally substituted heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, optionally substituted heteroaliphatic, optionally substituted aryl, optionally substituted heteroaryl, —C(═O)R^{c1}, C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted heterocyclic or heteroaryl ring or the group ≡C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring;

wherein each instance of R^{x} is, independently, hydrogen, an optionally substituted aliphatic, optionally substituted alkoxy, optionally substituted heteroaliphatic, optionally substituted aryloxy, optionally substituted heteroaryloxy, optionally substituted aryl, or optionally substituted heteroaryl group;

when W is —C— or —C(R^{d})— then:
(i) Z is a bond, —O—, —S—, —C(R^{d})₂—, —C(R^{d})═C(R^{d}), —C(R^{d})═N—, or —N(R^{e})—;

or
(ii) Z is —N— joined via a linker group -L- to the group R^{L1} to form a 5- to 7-membered palladacycle, wherein -L- is selected from absent, —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, or an —N(R^{c})₂ group wherein two R^{c} groups are joined to form an optionally substituted heterocyclic or heteroaryl ring;

or
(iii) Z is —N—S(O)₂—R^{e3} and the linker group -L- is absent;

or

when W is —N— or —N(R^{e})—, then Z is a bond, —C(R^{d})₂—, —C(R^{d})═C(R^{d})—, or —C(R^{d})═N—;

or

when W is —SO₂— or ═N—, then R₄ is absent;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, C(═NR^{e3})OR^{e2}, —C(═NR^{e3})N(R^{e3})₂, —S(O)₂R^{e1}, —S(O)R^{e1}, a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted heterocyclic or heteroaryl ring;

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

R² and R³ are optionally joined to form an optionally substituted 5- to 7-membered heteroaryl, aryl, heterocyclic or carbocyclic ring;

R³ and R⁴ are optionally joined to form an optionally substituted 5- to 7-membered heteroaryl, aryl, heterocyclic or carbocyclic ring,

wherein the each of curved dotted lines

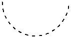

independently represents optional ioining of an optionally substituted 5- to 7-membered ring, and

wherein [symbol] represents a single or double bond.

In some embodiments, R¹ and R² are joined to form an optionally substituted 5- to 6-membered heteroaryl, aryl, heterocyclic or carbocyclic ring. In some embodiments, R¹ and R² are joined to form an optionally substituted 5-membered heteroaryl, aryl, heterocyclic or carbocyclic ring. In some embodiments, R¹ and R² are joined to form an optionally substituted 6-membered heteroaryl, aryl, heterocyclic or carbocyclic ring.

In some embodiments, R² and R³ are joined to form an optionally substituted 5- to 6-membered heteroaryl, aryl, heterocyclic or carbocyclic ring. In some embodiments, R² and R³ are joined to form an optionally substituted 5-membered heteroaryl, aryl, heterocyclic or carbocyclic ring. In some embodiments, R² and R³ are joined to form an optionally substituted 6-membered heteroaryl, aryl, heterocyclic or carbocyclic ring.

In some embodiments, R³ and R⁴ are joined to form an optionally substituted 5- to 6-membered heteroaryl, aryl, heterocyclic or carbocyclic ring. In some embodiments, R³ and R⁴ are joined to form an optionally substituted 5-membered heteroaryl, aryl, heterocyclic or carbocyclic ring. In some embodiments, R³ and R⁴ are joined to form an optionally substituted 6-membered heteroaryl, aryl, heterocyclic or carbocyclic ring.

Any of the optionally substituted 5- to 6-membered heteroaryl, aryl, heterocyclic or carbocyclic rings formed by joining R¹ and R², R² and R³ and/or R³ and R⁴ can be, for example, an optionally substituted 5- to 6-membered heteroaryl, an optionally substituted 6-membered aryl, an optionally substituted 5- to 6-membered heterocyclic or an optionally substituted 5- to 6-membered carbocyclic ring.

Exemplary 5-membered heteroaryl rings include, but are not limited to, optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isooxazolyl, optionally substituted oxadiaziolyl or optionally substituted oxadiaziolyl ring.

Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

Exemplary 5-membered heterocyclic rings include, but are not limited to, optionally substituted pyrrolidinyl, optionally substituted tetrahydrofuranyl, optionally substituted tetrahydrothiophenyl, and optionally substituted 1,3 dithiolanyl.

Exemplary 6-membered heterocyclic rings include, but are not limited to, optionally substituted piperidinyl, optionally substituted piperazinyl, optionally substituted morpholinyl, optionally substituted tetrahydropyranyl and optionally substituted dioxanyl.

Exemplary 5-membered carbocyclic rings include, but are not limited to, optionally substituted cyclopentyl and optionally substituted cyclopentenyl.

Exemplary 6-membered carbocyclic rings include, but are not limited to, optionally substituted cyclohexyl and optionally substituted cyclohexenyl.

In some embodiments, R² and R³ are not joined together to form a cyclic structure.

In some embodiments, R³ and R⁴ are not joined together to form a cyclic structure.

In some embodiments, both R¹ and R² and R² and R³ are joined to form rings, but R³ and R⁴ are not joined together to form a cyclic structure.

In some embodiments, both R¹ and R² and R³ and R⁴ are joined to form rings, but R² and R³ are not joined together to form a cyclic structure.

In some embodiments, both R² and R³ and R³ and R⁴ are joined to form rings, but R¹ and R² are not joined together to form a cyclic structure.

Palladium (II) Complexes with Bidentate Ligand

In some embodiments, Z is not joined via a linker group -L- to the group R^{L1} to form a 5- to 7-membered palladacycle.

For example, in some embodiments, the palladium (II) complex comprises a bidentate ligand. In some embodiments, the palladium (II) complex is of the formula (I-a):

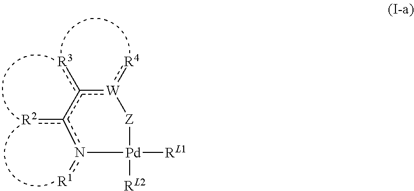

wherein Pd, [symbol] ,

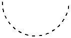

W, R^{L1}, R^{L2}, Z, R¹, R², R³ and R⁴ are as defined above and herein.

In some embodiments, R¹ and R² are joined to form an optionally substituted 6-membered pyridinyl ring to provide a palladium (II) complex of the formula (I-b):

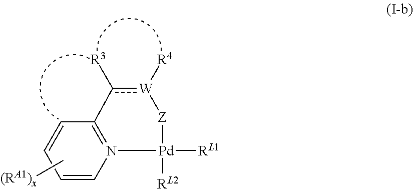

wherein

Pd, [symbol] ,

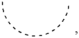

W, R^{L1}, R^{L2}, Z, R³, and R⁴ are as defined above and herein;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, —SR^{A1b}, —N(R^{A1c})₂, —C(═O)R^{A1d}, —C(═O)OR^{A1a}, —C(═O)N(R^{A1c})₂, —C(═NR^{A1c})R^{A1d}, —C(═NR^{A1c})OR^{A1a}, —C(NR^{A1c})N(R^{A1c})₂, —S(O)₂R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group; and

x is an integer between 0-4, inclusive.

In some embodiments, each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}. In some embodiments, each instance of R^{A1} is, independently, hydrogen, halogen, optionally substituted C₁₋₆ alkyl, —NO₂, —CF₃, or —OR^{A1a}. In some embodiments, each instance of R^{A1} is, independently, hydrogen, —CH₃, -tBu, —CN, —NO₂, —CF₃, or —OCH₃. In some embodiments, each instance of R^{A1} is hydrogen.

In some embodiments, R³ and R⁴ are joined to form an optionally substituted aryl ring to provide a palladium (II) complex of the formula (I-c):

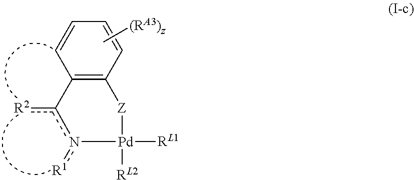

wherein

Pd, [symbol] ,

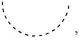

R¹, R², R^{L1}, R^{L2}, and Z are as defined above and herein;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —C(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3c} is, independently, hydrogen, an optionally substituted aliphatic, optionally substituted heteroaliphatic, optionally substituted aryl, optionally substituted heteroaryl or a suitable amino protecting group, or two R^{A3c} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group; and

z is an integer between 0-3, inclusive.

In some embodiments, each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}. In some embodiments, each instance of R^{A3} is, independently, hydrogen, halogen, optionally substituted C₁₋₆ alkyl, —NO₂, —CF₃, or —OR^{A3a}. In some embodiments, each instance of R^{A3} is, independently, hydrogen, —CH₃, -tBu, —CN, —NO₂, —CF₃, or —OCH₃. In some embodiments, each instance of R^{A3} is hydrogen.

In some embodiments, R¹ and R² are joined to form an optionally substituted 6-membered pyridinyl ring and R³ and R⁴ are joined to form an optionally substituted aryl ring to provide a palladium (II) complex of the formula (I-d):

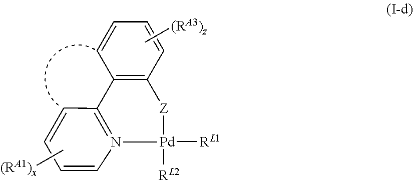

wherein Pd, [symbol] ,

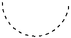

R^{A1}, R^{A3}, R^{L1}, R^{L2}, x, z, and Z are as defined above and herein.

In some embodiments, R¹ and R² are joined to form an optionally substituted 6-membered pyridinyl ring and R² and R³ are joined to form an optionally substituted 6-membered aryl ring, to provide a palladium (II) catalyst of the formula (I-e):

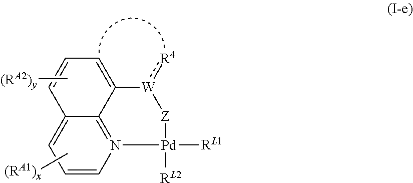

wherein

Pd, [symbol] ,

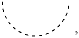

W, R^{A1}, R^{L1}, R^{L2}, R⁴, x, and Z are as defined above and herein;

each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}, —SR^{A2b}, —N(R^{A2c})₂, —C(═O)R^{A2dd}, —C(═O)OR^{A2a}, —C(═O)N(R^{A2c})₂, —C(═NR^{A2c})R^{A2d}, C(═NR^{A2c})OR^{A2a}, —C(═NR^{A2c})N(R^{A2c})₂, —S(O)₂R^{A2d}, —S(O)R^{A2d}, or two R^{A2} groups adjacent to each other are joined to form a 5- to 6-membered aryl, heteroaryl, heterocyclic or carbocyclic ring, wherein R^{A2a} is hydrogen, an optionally substituted aliphatic, optionally substituted heteroaliphatic, optionally substituted aryl, optionally substituted heteroaryl or a suitable hydroxyl protecting group; wherein R^{A2b} is hydrogen, an optionally substituted aliphatic, optionally substituted heteroaliphatic, optionally substituted aryl, optionally substituted heteroaryl or a suitable thiol protecting group; wherein each R^{A2c} is, independently, hydrogen, an optionally substituted aliphatic, optionally substituted heteroaliphatic, optionally substituted aryl, optionally substituted heteroaryl or a suitable amino protecting group, or two R^{A2c} groups are joined together to form a heterocyclic or heteroaryl group; and wherein each R^{A2d} is, independently, an optionally substituted aliphatic, optionally substituted heteroaliphatic, optionally substituted aryl, or an optionally substituted heteroaryl group; and

y is an integer between 0-2, inclusive.

In some embodiments, each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}. In some embodiments, each instance of R^{A2} is, independently, hydrogen, halogen, optionally substituted C₁₋₆ alkyl, —NO₂, —CF₃, or —OR^{A2a}. In some embodiments, each instance of R^{A2} is, independently, hydrogen, —CH₃, -tBu, —CN, —NO₂, —CF₃, or —OCH₃. In some embodiments, each instance of R^{A2} is hydrogen.

In some embodiments, R² and R³ are joined to form an optionally substituted 6-membered aryl ring to provide a palladium (II) catalyst of the formula (I-f):

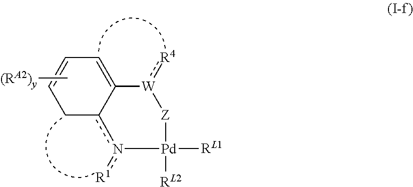

wherein Pd, [symbol] ,

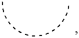

W, R^{A2}, R¹, R⁴, R^{L1}, R^{L2}, y and Z are as defined above and herein.

In some embodiments, R¹ and R² are joined to form an optionally substituted pyridinyl ring, R² and R³ are joined to form an optionally substituted 6-membered aryl ring and R³ and R⁴ are joined to form an optionally substituted 6-membered aryl ring to form the bidentate palladium (II) complex of the formula (I-g):

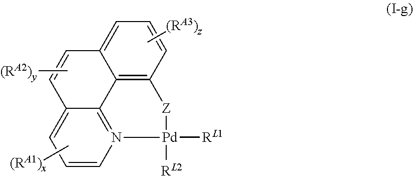

wherein Pd, R^{L1}, R^{L2}, Z, R^{A1}, R^{A2}, R^{A3}, x, y and z are as defined above and herein.

In some embodiments, wherein R² and R³ are not joined to form an optionally substituted 5- to 6-membered ring, the palladium (II) complex is of the formula (I-h):

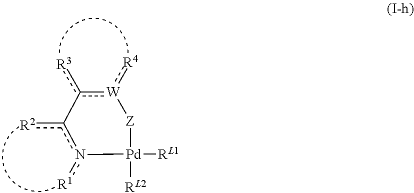

wherein Pd, [symbol] ,

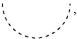

W, Z, R¹, R², R³, R⁴, R^{L1} and R^{L2} are as defined above and herein; and

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring; and

R³ and R⁴ are optionally joined to form an optionally substituted 5- to 7-membered heteroaryl, aryl, heterocyclic or carbocyclic ring.

In some embodiments, wherein R² and R³ are not joined to form a cyclic structure, the palladium (II) complex is of the formula (I-i):

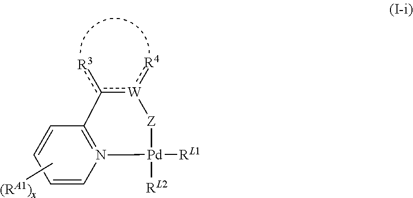

wherein Pd, [symbol] ,

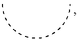

W, R³, R⁴, R^{L1}, R^{L2}, R^{A1} and x are as defined above and herein.

In some embodiments, wherein R² and R³ are not joined to form a cyclic structure, the palladium (II) complex is of the formula (I-j):

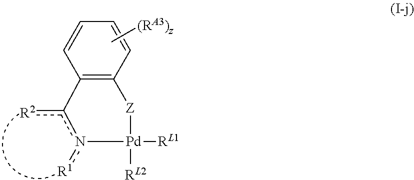

wherein Pd, [symbol] ,

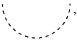

R¹, R², R^{L1}, R^{L2}, R^{A3}, Z, and z are as defined above and herein.

In some embodiments, wherein R² and R³ are not joined to form a cyclic structure, the palladium (II) complex is of the formula (I-k):

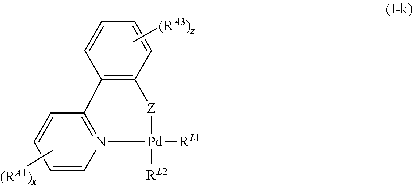

wherein Pd, R^{L1}, R^{L2}, R^{A1}, R^{A3}, Z, z and x are as defined above and herein.

In some embodiments, in any of the above formulae Z is a bond. In other embodiments, Z is

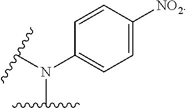

In other embodiments, Z is

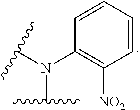

In some embodiments, wherein R² and R³ are not joined to form a cyclic structure and Z is a bond, the palladium (II) complex is of the formula (I-l):

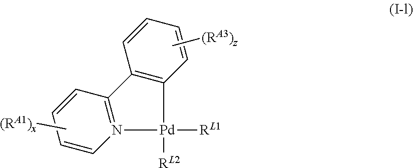

wherein R^{L1}, R^{L2}, R^{A1}, R^{A3}, z and x are as defined above and herein.

In some embodiments, the palladium (II) complex is of the formula (I-k):

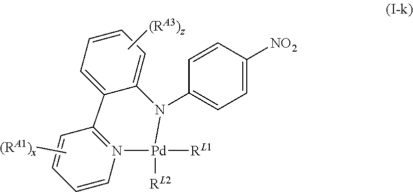

wherein R^{L1}, R^{L2}, R^{A1}, R^{A3}, z, and x are as defined above and herein.

In some embodiments, the palladium (II) complex is of the formula (I-l′):

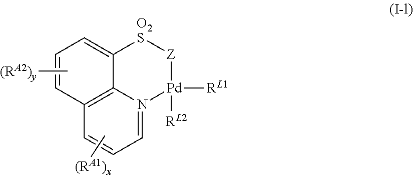

wherein Pd, R^{L1}, R^{L2}, R^{A1}, R^{A2}, x, y, and Z are as defined above and herein.

In some embodiments, the palladium (II) complex is of the formula (I-m′):

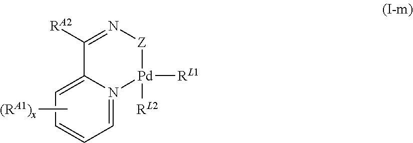

wherein Pd, R^{L1}, R^{L2}, R^{A1}, R^{A2}, x, and Z are as defined above and herein.

In some embodiments, the palladium (II) complex is of the formula (I-n′):

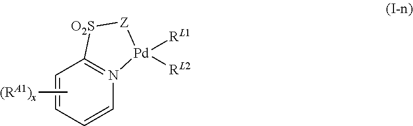

wherein Pd, R^{L1}, R^{L2}, R^{A1}, x, and Z are as defined above and herein.

Palladium (II) Complexes with Tridentate Ligand

In some embodiments, Z is joined via a linker group -L- to the group R^{L1} to form a 5- to 7-membered palladacycle.

In some embodiments, the palladium (II) catalyst comprises a tridentate ligand. In some embodiments, the palladium (II) catalyst of the formula (I-a′):

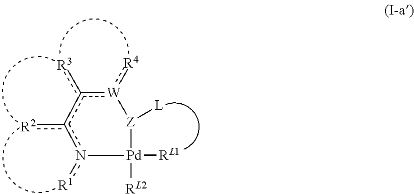

wherein

Pd, [symbol] ,

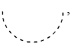

W, R^{L1}, R^{L2}, R¹, R², R³, and R⁴ are as defined above and herein;

Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, or an —N(R^{c})₂ group wherein two R^{c} groups are joined to form an optionally substituted heterocyclic or heteroaryl ring; and

the curved solid line

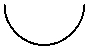

represents joining of the 5- to 7-membered palladacycle.

In some embodiments, R¹ and R² are joined to form an optionally substituted 6-membered pyridinyl ring to provide a palladium (II) complex of the formula (I-b′):

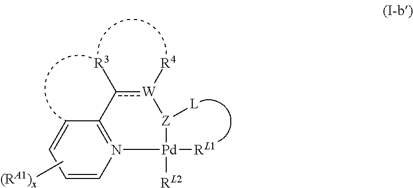

wherein

Pd, [symbol] ,

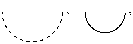

W, L, R^{L1}, R^{L2}, Z, R³ and R⁴ are as defined above and herein;

each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}, SR^{A1b}, N(R^{A1c})₂, —C(═O)R^{A1d}, —C(═O)OR^{A1a}, —C(═O)N(R^{A1c})₂, —C(═NR^{A1c})R^{A1d}, —C(NR^{A1c})OR^{A1a}, —C(NR^{A1c})N(R^{A1c})₂, —S(O)₂R^{A1d}, —S(O)R^{A1d}, or two R^{A1} groups adjacent to each other are joined to form a 5- to 6-membered aryl, heteroaryl, heterocyclic or carbocyclic ring, wherein R^{A1a} is hydrogen, an optionally substituted aliphatic, optionally substituted heteroaliphatic, optionally substituted aryl, optionally substituted heteroaryl or a suitable hydroxyl protecting group; wherein R^{A1b} is hydrogen, an optionally substituted aliphatic, optionally substituted heteroaliphatic, optionally substituted aryl, optionally substituted heteroaryl or a suitable thiol protecting group; wherein each R^{A1c} is, independently, hydrogen, an optionally substituted aliphatic, optionally substituted heteroaliphatic, optionally substituted aryl, optionally substituted heteroaryl or a suitable amino protecting group, or two R^{A1c} groups are joined together to form a heterocyclic or heteroaryl group; and wherein each R^{A1d} is, independently, an optionally substituted aliphatic, optionally substituted heteroaliphatic, optionally substituted aryl, or an optionally substituted heteroaryl group; and

x is an integer between 0-4, inclusive.

In some embodiments, each instance of R^{A1} is, independently, hydrogen, halogen, optionally substituted aliphatic, optionally substituted heteroaliphatic, optionally substituted aryl, optionally substituted heteroaryl, —CN, —NO₂, —NC, —OR^{A1a}. In some embodiments, each instance of R^{A1} is, independently, hydrogen, halogen, optionally substituted C₁₋₆ alkyl, —NO₂, —CF₃, or —OR^{A1a}. In some embodiments, each instance of R^{A1} is, independently, hydrogen, —CH₃, -tBu, —CN, —NO₂, —CF₃, or —OCH₃. In some embodiments, each instance of R^{A1} is hydrogen.

In some embodiments, R³ and R⁴ are joined to form an optionally substituted aryl ring to provide a palladium (II) complex of the formula (I-c′):

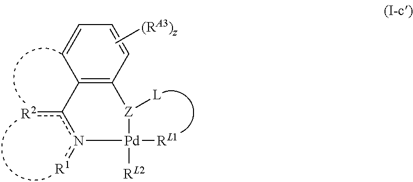

wherein

Pd, [symbol] ,

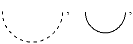

L, R¹, R², R^{L1}, R^{L2}, z, and Z are as defined above and herein;

each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}, —SR^{A3b}, —N(R^{A3c})₂, —C(═O)R^{A3d}, —C(═O)OR^{A3a}, —C(═O)N(R^{A3c})₂, —C(═NR^{A3c})R^{A3d}, —C(═NR^{A3c})OR^{A3a}, —C(═NR^{A3c})N(R^{A3c})₂, —S(O)₂R^{A3d}, —S(O)R^{A3d}, or two R^{A3} groups adjacent to each other are joined to form a 5- to 6-membered aryl, heteroaryl, heterocyclic or carbocyclic ring, wherein R^{A3a} is hydrogen, an optionally substituted aliphatic, optionally substituted heteroaliphatic, optionally substituted aryl, optionally substituted heteroaryl or a suitable hydroxyl protecting group; wherein R^{A3b} is hydrogen, an optionally substituted aliphatic, optionally substituted heteroaliphatic, optionally substituted aryl, optionally substituted heteroaryl or a suitable thiol protecting group; wherein each R^{A3c} is, independently, hydrogen, an optionally substituted aliphatic, optionally substituted heteroaliphatic, optionally substituted aryl, optionally substituted heteroaryl or a suitable amino protecting group, or two R^{A3C} groups are joined together to form a heterocyclic or heteroaryl group; and wherein each R^{A3d} is, independently, an optionally substituted aliphatic, optionally substituted heteroaliphatic, optionally substituted aryl, or an optionally substituted heteroaryl group; and

z is an integer between 0-3, inclusive.

In some embodiments, each instance of R^{A3} is, independently, hydrogen, halogen, optionally substituted aliphatic, optionally substituted heteroaliphatic, optionally substituted aryl, optionally substituted heteroaryl, —CN, —NO₂, —NC, —OR^{A3a}. In some embodiments, each instance of R^{A3} is, independently, hydrogen, halogen, optionally substituted C₁₋₆ alkyl, —NO₂, —CF₃, or —OR^{A3a}. In some embodiments, each instance of R^{A3} is, independently, hydrogen, —CH₃, -tBu, —CN, —NO₂, —CF₃, or —OCH₃. In some embodiments, each instance of R^{A3} is hydrogen.

In some embodiments, R¹ and R² are joined to form an optionally substituted 6-membered pyridinyl ring and R³ and R⁴ are joined to form an optionally substituted aryl ring to provide a palladium (II) complex of the formula (I-d′):

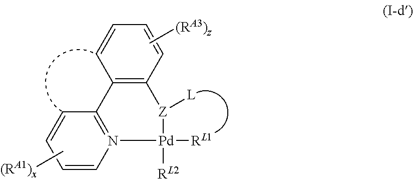

wherein Pd, [symbol] ,

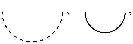

L, R^{A1}, R^{A3}, R^{L1}, R^{L2}, x, z, and Z are as defined above and herein.

In some embodiments, R¹ and R² are joined to form an optionally substituted 6-membered pyridinyl ring and R² and R³ are joined to form an optionally substituted 6-membered aryl ring, to provide a palladium (II) catalyst of the formula (I-e′):

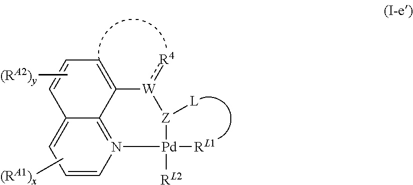

wherein Pd, [symbol] ,

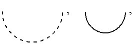

L, W, R^{A1}, R^{L1}, R_{L2}, R⁴, x and Z are as defined above and herein;

each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}, —SR^{A2b}, —N(R^{A2c})₂, —C(═O)R^{A2d}, —C(═O)OR^{A2a}, —C(═O)N(R^{A2c})₂, —C(═NR^{A2c})R^{A2d}, —C(═NR^{A2c})OR^{A2a}, —C(═NR^{A2c})N(R^{A2c})₂, —S(O)₂R^{A2d}, —S(O)R^{A2d}, or two R^{A2} groups adjacent to each other are joined to form a 5- to 6-membered aryl, heteroaryl, heterocyclic or carbocyclic ring, wherein R^{A2a} is hydrogen, an optionally substituted aliphatic, optionally substituted heteroaliphatic, optionally substituted aryl, optionally substituted heteroaryl or a suitable hydroxyl protecting group; wherein R^{A2b} is hydrogen, an optionally substituted aliphatic, optionally substituted heteroaliphatic, optionally substituted aryl, optionally substituted heteroaryl or a suitable thiol protecting group; wherein each R^{A2c} is, independently, hydrogen, an optionally substituted aliphatic, optionally substituted heteroaliphatic, optionally substituted aryl, optionally substituted heteroaryl or a suitable amino protecting group, or two R^{A2c} groups are joined together to form a heterocyclic or heteroaryl group; and wherein each R^{A2d} is, independently, an optionally substituted aliphatic, optionally substituted heteroaliphatic, optionally substituted aryl, or an optionally substituted heteroaryl group; and

y is an integer between 0-2, inclusive.

In some embodiments, each instance of R^{A2} is, independently, hydrogen, halogen, optionally substituted aliphatic, optionally substituted heteroaliphatic, optionally substituted aryl, optionally substituted heteroaryl, —CN, —NO₂, —NC, —OR^{A2a}. In some embodiments, each instance of R^{A2} is, independently, hydrogen, halogen, optionally substituted C₁₋₆ alkyl, —NO₂, —CF₃, or —OR^{A2a}. In some embodiments, each instance of R^{A2} is, independently, hydrogen, —CH₃, -tBu, —CN, —NO₂, —CF₃, or —OCH₃. In some embodiments, each instance of R^{A2} is hydrogen.

In some embodiments, R² and R³ are joined to form an optionally substituted 6-membered aryl ring to provide a palladium (II) catalyst of the formula (I-f′):

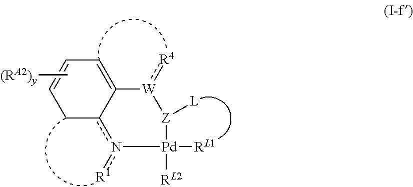

wherein Pd, [symbol] ,

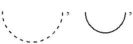

L, W, R^{A2}, R¹, R⁴, R^{L1}, R^{L2}, y and Z are as defined above and herein.

In some embodiments, R¹ and R² are joined to form an optionally substituted pyridinyl ring, R² and R³ are joined to form an optionally substituted 6-membered aryl ring and R³ and R⁴ are joined to form an optionally substituted 6-membered aryl ring to form the palladium (II) complex of the formula (I-g′):

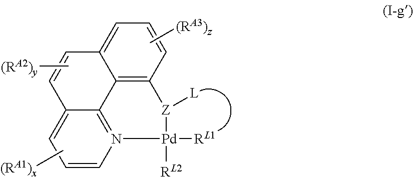

wherein

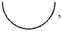

L, R^{L1}, R^{L2}, Z, R^{A1}, R^{A2}, R^{A3}, y and z are as defined above and herein.

In some embodiments, wherein R² and R³ are not joined to form an optionally substituted 5- to 6-membered ring, the palladium (II) complex is of the formula (I-h′):

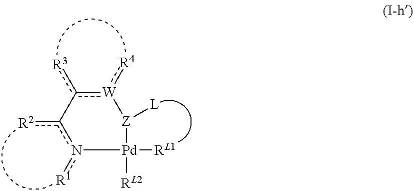

wherein Pd, [symbol] ,

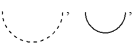

L, W, Z, R¹, R², R³, R⁴, R^{L1} and R^{L2} are as defined above and herein; and

R¹, R², R³ and R⁴ are, independently, an optionally substituted aliphatic, optionally substituted heteroaliphatic, optionally substituted aryl, optionally substituted heteroaryl group,

R¹ and R² are optionally joined to form an optionally substituted 5- to 7-membered heteroaryl, aryl, heterocyclic or carbocyclic ring;

and

R³ and R⁴ are optionally joined to form an optionally substituted 5- to 7-membered heteroaryl, aryl, heterocyclic or carbocyclic ring.

In some embodiments, wherein R² and R³ are not joined to form a cyclic structure, the palladium (II) complex is of the formula (I-i′):

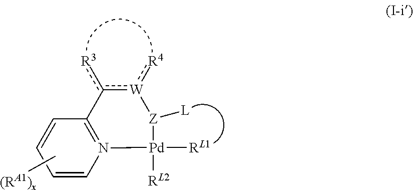

wherein Pd, [symbol] ,

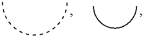

L, W, R³, R⁴, R^{L1}, R^{L2}, R^{A1} and x are as defined above and herein.

In some embodiments, wherein R² and R³ are not joined to form a cyclic structure, the palladium (II) complex is of the formula (I-j′):

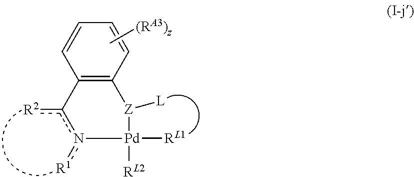

wherein Pd, [symbol] ,

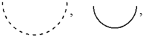

L, R¹, R², R^{L1}, R^{L2}, R^{A3} and z are as defined above and herein.

In some embodiments, wherein R² and R³ are not joined to form a cyclic structure, the palladium (II) complex is of the formula (I-k′):

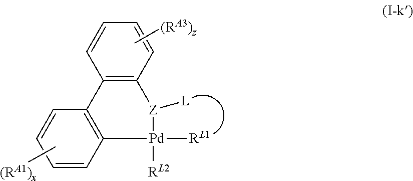

wherein Pd,

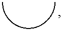

L, R^{L1}, R^{L2}, R^{A1}, R^{A3}, Z, z and x are as defined above and herein.

Groups R^{L1} and R^{L2}

As defined generally above, R^{L1} and R^{L2} are, independently, halogen, optionally substituted aliphatic, optionally substituted heteroaliphatic, optionally substituted aryl, optionally substituted heteroaryl, —OR^{a}, —SR^{b}, —N(R^{c})₃, —N(R^{c})₂, or —P(R^{x})₃,

wherein each instance of R^{a} is, independently, hydrogen, an optionally substituted aliphatic, optionally substituted heteroaliphatic, optionally substituted aryl, optionally substituted heteroaryl, —C(═O)R^{a1}, —C(═O)OR^{a2}, —C(═O)N(R^{a3})₂, —C(═NR^{a3})R^{a3}, —C(═NR^{a3})OR^{a1}, —C(═NR^{a3})N(R^{a3})₂, —S(O)₂R^{a1}, —S(O)R^{a1}, or a suitable hydroxyl protecting group, wherein R^{a1} is an optionally substituted aliphatic, optionally substituted heteroaliphatic, optionally substituted aryl or optionally substituted heteroaryl group; wherein R^{a2} is an optionally substituted aliphatic, optionally substituted heteroaliphatic, optionally substituted aryl, optionally substituted heteroaryl group, or a suitable hydroxyl protecting group; wherein R^{a3} is an optionally substituted aliphatic, optionally substituted heteroaliphatic, optionally substituted aryl, optionally substituted heteroaryl group, or a suitable amino protecting group, or two R^{a3} groups are joined to form an optionally substituted heterocyclic or heteroaryl ring;

wherein each instance of R^{b} is, independently, an optionally substituted aliphatic, heteroaliphatic, aryl, heteroaryl, —C(═O)R^{b1}, —C(═O)OR^{b2}, —C(═O)N(R^{b3})₂, —C(═NR^{b3})R^{b3}, —C(═NR^{b3})OR^{b1}, —C(═NR^{a3})N(R^{b3})₂, or a suitable thiol protecting group, wherein R^{b1} is an optionally substituted aliphatic, optionally substituted heteroaliphatic, optionally substituted aryl or optionally substituted heteroaryl group; wherein R^{b2} is an optionally substituted aliphatic, optionally substituted heteroaliphatic, optionally substituted aryl, optionally substituted heteroaryl group, or a suitable hydroxyl protecting group; wherein R^{b3} is an optionally substituted aliphatic, optionally substituted heteroaliphatic, optionally substituted aryl, optionally substituted heteroaryl group, or a suitable amino protecting group, or two R^{b3} groups are joined to form an optionally substituted heterocyclic or heteroaryl ring;

wherein each instance of R^{c} is, independently, hydrogen, an optionally substituted aliphatic, heteroaliphatic, aryl, heteroaryl, —C(═O)R^{c1}, —C(═O)OR^{c2}, —C(═O)N(R^{c3})₂, —C(═NR^{c3})R^{c3}, —C(═NR^{c3})OR^{c1}, —C(═NR^{c3})N(R^{c3})₂, —S(O)₂R^{c1}, —S(O)R^{c1}, or a suitable amino protecting group, or two R^{c} groups are joined to form an optionally substituted 5- to 6-membered heterocyclic or heteroaryl ring or the group ≡C(R^{c1}), wherein R^{c1} is an optionally substituted aliphatic, optionally substituted heteroaliphatic, optionally substituted aryl or optionally substituted heteroaryl group; wherein R^{c2} is an optionally substituted aliphatic, optionally substituted heteroaliphatic, optionally substituted aryl, optionally substituted heteroaryl group, or a suitable hydroxyl protecting group; wherein R^{c3} is an optionally substituted aliphatic, optionally substituted heteroaliphatic, optionally substituted aryl, optionally substituted heteroaryl group, or a suitable amino protecting group, or two R^{c3} groups are joined to form an optionally substituted heterocyclic or heteroaryl ring; and

wherein each instance of R^{x} is, independently, hydrogen, an optionally substituted aliphatic, optionally substituted alkoxy, optionally substituted heteroaliphatic, optionally substituted aryloxy, optionally substituted heteroaryloxy, optionally substituted aryl, or optionally substituted heteroaryl group.

In some embodiments, at least one of R^{L1} and R^{L2} is selected from halogen, —OR^{a}, —SR^{b}, —N(R^{c})₃, —N(R^{c})₂, or —P(R^{x})₃. In some embodiments, both R^{L1} and R^{L2} are, independently, selected from halogen, —OR^{a}, —SR^{b}, —N(R^{c)}₃, —N(R^{c})₂, or —P(R^{x})₃.

In some embodiments, R^{L1} is halogen, —OR^{a}, —SR^{b}, or —N(R^{c})₂ and R^{L2} is —N(R^{c})₂. In some embodiments, R^{L1} is halogen, —OR^{a} or —N(R^{c})₂, and R^{L2} is —N(R^{c})₂. In some embodiments, R^{L1} is halogen or —OR^{a}, and R^{L2} is —N(R^{c})₂. In some embodiments, R^{L1} is and R^{L2} is —N(R^{c})₂.) In some embodiments, R^{L1} is halogen and R^{L2} is —N(R^{c})₂. In some embodiments, R^{L1} is —OR^{a} and R^{L2} is —N(R^{c})₂. In some embodiments, both R^{L1} and R^{L2} are independently —N(R^{c})₂.

In some embodiments, R^{L1} is halogen. In some embodiments, R^{L1} is —Cl. In some embodiments, R^{L1} is —Br. In some embodiments, R^{L1} is —I. In some embodiments, R^{L1} is —F.

In some embodiments, R^{L1} is —OR^{a}.

In some embodiments, R^{L}1 is —OC(═O)R^{a1} wherein R^{a1} is an optionally substituted aliphatic, optionally substituted heteroaliphatic, optionally substituted aryl or optionally substituted heteroaryl group. In some embodiments, R^{L1} is —OC(═O)R^{a1} wherein R^{a1} is an optionally substituted aliphatic group. In some embodiments, R^{L1} is —OC(═O)R^{a1} wherein R^{a1} is an optionally substituted C₁₋₆ alkyl group. In some embodiments, R^{L1} is —OC(═O)R^{a1} wherein R^{a1} is an optionally substituted C₁₋₄ alkyl group. In some embodiments, R^{L1} is —OC(═O)R^{a1} wherein R^{a1} is an optionally substituted C₁₋₂ alkyl group. In some embodiments, R^{L1} is —OC(═O)CH₃.

In some embodiments, R^{L1} is —P(R^{X})₃.

In some embodiments, R^{L2} is —N(R^{c})₂.

In some embodiments, R^{L2} is —N(R^{c})₂ wherein two R^{c} groups are joined to form the group ≡C(R^{c1}), wherein R^{c1} is an optionally substituted aliphatic, optionally substituted heteroaliphatic, optionally substituted aryl or optionally substituted heteroaryl group. In some embodiments, R^{L2} is —N(R^{c})₂ wherein two R^{c} groups are joined to form the group ≡C(R^{c1}), wherein R^{c1} is an optionally substituted aliphatic group. In some embodiments, R^{L2} is —N(R^{c})₂ wherein two R^{c} groups are joined to form the group ≡C(R^{c1}), wherein R^{c1} is an optionally substituted C₁₋₆ alkyl group. In some embodiments, R^{L2} is —N(R^{c})₂ wherein two R^{c} groups are joined to form the group ≡C(CH₃) or ≡C(CH₂Ph).

In some embodiments, R^{L2} is —N(R^{c})₂ wherein two R^{c} groups are joined to form an optionally substituted heterocyclic or heteroaryl ring.

In some embodiments, R^{L2} is —N(R^{c})₂ wherein two R^{c} groups are joined to form an optionally substituted 5- to 6-membered heterocyclic or heteroaryl ring.

In some embodiments, R^{L2} is —N(R^{c})₂ wherein two R^{c} groups are joined to form an optionally substituted 5-membered heterocyclic ring. Exemplary 5-membered heterocyclic rings include, but are not limited to, an optionally substituted pyrrolidinyl ring.

In some embodiments, R^{L2} is —N(R^{c})₂ wherein two R^{c} groups are joined to form an optionally substituted 5-membered heteroaryl ring. Exemplary 5-membered heteroaryl rings include, but are not limited to, an optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isooxazolyl, optionally substituted oxadiaziolyl or optionally substituted oxadiaziolyl ring.

In some embodiments, R^{L2} is —N(R^{c})₂ wherein two R^{c} groups are joined to form an optionally substituted 6-membered heterocyclic ring. Exemplary 6-membered heterocyclic rings include, but are not limited to, optionally substituted piperidinyl, optionally substituted piperazinyl or optionally substituted morpholinyl ring.

In some embodiments, R^{L2} is —N(R^{c})₂ wherein two R^{c} groups are joined to form an optionally substituted 6-membered heteroaryl ring. Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

In some embodiments, R^{L2} is an optionally substituted pyridinyl ring.

In some embodiments, R^{L1} is —N(R^{c})₂.

In some embodiments, R^{L1} is —N(R^{c})₂ wherein two R^{c} groups are joined to form the group ≡C(R^{c1}), wherein R^{c1} is an optionally substituted aliphatic, optionally substituted heteroaliphatic, optionally substituted aryl or optionally substituted heteroaryl group. In some embodiments, R^{L1} is —N(R^{c})₂ wherein two R^{c} groups are joined to form the group ≡C(R^{c1}), wherein R^{c1} is an optionally substituted aliphatic group. In some embodiments, R^{L1} is —N(R^{c})₂ wherein two R^{c} groups are joined to form the group ≡C(R^{c1}), wherein R^{c1} is an optionally substituted C₁₋₆ alkyl group. In some embodiments, R^{L1} is —N(R^{c})₂ wherein two R^{c} groups are joined to form the group ≡C(CH₃) or ≡C(CH₂Ph).

In some embodiments, R^{L1} is —N(R^{c})₂ wherein two R^{c} groups are joined to form an optionally substituted 5- to 6-membered heterocyclic or heteroaryl ring.

In some embodiments, R^{L1} is —N(R^{c})₂ wherein two R^{c} groups are joined to form an optionally substituted 5-membered heterocyclic ring. Exemplary 5-membered heterocyclic rings are provided above and herein.

In some embodiments, R^{L1} is —N(R^{c})₂ wherein two R^{c} groups are joined to form an optionally substituted 5-membered heteroaryl ring. Exemplary 5-membered heteroaryl rings are provided above and herein.

In some embodiments, R^{L1} is —N(R^{c})₂ wherein two R^{c} groups are joined to form an optionally substituted 6-membered heterocyclic ring. Exemplary 6-membered heterocyclic rings are provided above and herein.

In some embodiments, R^{L1} is —N(R^{c})₂ wherein two R^{c} groups are joined to form an optionally substituted 6-membered heteroaryl ring. Exemplary 6-membered heteroaryl rings are provided above and herein.

In some embodiments, R^{L1} is an optionally substituted pyridinyl ring.

Optionally substituted pyridinyl rings include, but are not limited to, rings of the formula

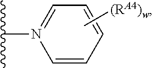

wherein each instance of R^{A4} is, independently, hydrogen, halogen, optionally substituted aliphatic, optionally substituted heteroaliphatic, optionally substituted aryl, optionally substituted heteroaryl, —CN, —NO₂, —NC, —OR^{A4a}, —SR^{A4b}, —N(R^{A4c})₂, —C(═O)R^{A4d}, —C(═O)OR^{A4a}, —C(═O)N(R^{A4c})₂, —C(═NR^{A4c})R^{A4d}, —C(═NR^{A4c})OR^{A4a}, —C(═NR^{A4c})N(R^{A4c})₂, —S(O)₂R^{A4d}, —S(O)R^{A4d}, or two R^{A4} groups adjacent to each other are joined to form a 5- to 6-membered aryl, heteroaryl, heterocyclic or carbocyclic ring, wherein R^{A4a} is hydrogen, an optionally substituted aliphatic, optionally substituted heteroaliphatic, optionally substituted aryl, optionally substituted heteroaryl or a suitable hydroxyl protecting group; wherein R^{A4b} is hydrogen, an optionally substituted aliphatic, optionally substituted heteroaliphatic, optionally substituted aryl, optionally substituted heteroaryl or a suitable thiol protecting group; wherein each R^{A4c} is, independently, hydrogen, an optionally substituted aliphatic, optionally substituted heteroaliphatic, optionally substituted aryl, optionally substituted heteroaryl or a suitable amino protecting group, or two R^{A4c} groups are joined together to form a heterocyclic or heteroaryl group; and wherein each R^{A4d} independently, an optionally substituted aliphatic, optionally substituted heteroaliphatic, optionally substituted aryl, or an optionally substituted heteroaryl group, and

w is an integer between 0 to 5, inclusive.

In some embodiments, the optionally substituted pyridinyl ring is of the formulae:

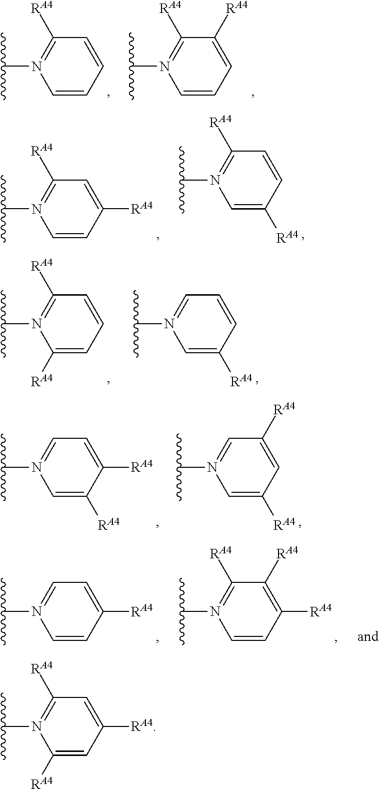

In some embodiments, the optionally substituted pyridinyl ring is:

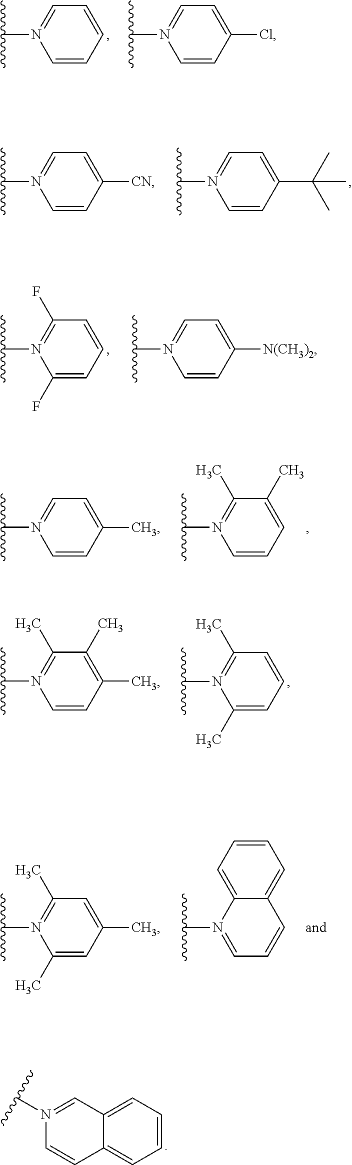

In some embodiments, R^{L2} is —P(R^{X})₃. In some embodiments, R^{X} is optionally substituted aliphatic. In some embodiments, R^{X} is optionally substituted aryl. In some embodiments, R^{X} is optionally substituted alkoxy. In some embodiments, R^{X} is optionally substituted aryloxy. In some embodiments, R^{L2} is —P(Me)₃. In some embodiments, R^{L2} is —P(Et)₃. In some embodiments, R^{L2} is —P(tert-Bu)₃. In some embodiments, R^{L2} is —P(Cy)₃. In some embodiments, R^{L2} is —P(Ph)₃. In some embodiments, R^{L2} is —PMe(Ph)₂. In some embodiments, R^{L2} is —PF₃. In some embodiments, R^{L2} is —P(OMe)₃. In some embodiments, R^{L2} is —P(OEt)₃. In some embodiments, R^{L2} is —P(OPh)₃.

Z, L, and R^{L1}

As generally defined above, in some embodiments, Z is —N— joined via a linker group -L- to the group R^{L1} to form a 5- to 7-membered palladacycle, wherein -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)— and R^{L1} is an optionally substituted aryl, optionally substituted heteroaryl, or an —N(R^{c})₂ group wherein two R^{c} groups are joined to form an optionally substituted membered heterocyclic or heteroaryl ring.

In some embodiments, R^{L1} is —N(R^{c})₂ optionally joined to Z via a linker group -L- to form a 5- to 7-membered palladacycle, wherein two R^{c} groups are joined to form an optionally substituted membered heterocyclic or heteroaryl ring.

In some embodiments, two R^{c} groups are joined to form an optionally substituted 5-membered heterocyclic ring. Exemplary 5-membered heterocyclic rings include, but are not limited to, an optionally substituted pyrrolidinyl ring.

In some embodiments, two R^{c} groups are joined to form an optionally substituted 5-membered heteroaryl ring. Exemplary 5-membered heteroaryl rings include, but are not limited to, an optionally substituted pyrrolyl, optionally substituted pyrazolyl, optionally substituted imidazolyl, optionally substituted triazolyl or optionally substituted tetrazolyl, optionally substituted thiazolyl, optionally substituted isothiazolyl, optionally substituted thiadiazolyl, optionally substituted oxazolyl, optionally substituted isooxazolyl, optionally substituted oxadiaziolyl or optionally substituted oxadiaziolyl ring.

In some embodiments, two R^{c} groups are joined to form an optionally substituted 6-membered heterocyclic ring. Exemplary 6-membered heterocyclic rings include, but are not limited to, optionally substituted piperidinyl, optionally substituted piperazinyl or optionally substituted morpholinyl ring.

In some embodiments, two R^{c} groups are joined to form an optionally substituted 6-membered heteroaryl ring. Exemplary 6-membered heteroaryl rings include, but are not limited to, optionally substituted pyridinyl, optionally substituted pyrimidinyl, optionally substituted pyrazinyl, optionally substituted pyridazinyl, optionally substituted triazinyl or optionally substituted tetrazinyl ring.

In some embodiments, two R^{c} groups are joined to form an optionally substituted bicyclic heteroaryl ring. Exemplary bicyclic heteroaryl rings include, but are not limited to, optionally substituted quinolinyl and optionally substituted isoquinolinyl.

In some embodiments, two R^{c} groups are joined to form an optionally substituted pyridinyl ring. In some embodiments, two R^{c} groups are joined to form an optionally substituted quinolinyl ring.

For example, in some embodiments, wherein two R^{c} groups are joined to form an optionally substituted pyridinyl ring, the group provided by Z, L and R^{L1} is of the formulae:

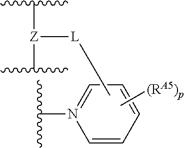

wherein:
Z is —N—;
L is -L- is selected from —C(═O)—, —C(═O)O—, —C(═O)N(R^{e3})—, —C(═NR^{e3})—, C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, —C(═NR^{A5a})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5C} groups are joined together to form a heterocyclic or heteroaryl group; and wherein each R^{A5d} is, independently, an optionally substituted aliphatic, optionally substituted heteroaliphatic, optionally substituted aryl, or an optionally substituted heteroaryl group, and

p is and integer between 0 to 5, inclusive.

In some embodiments, wherein two R^{c} groups are joined to form an optionally substituted quinolinyl ring, the group provided by Z, L and R^{L1} is of the formulae:

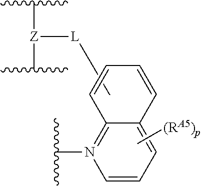

wherein:

Z is —N—;

L is -L- is selected from —C(═O)—, —C(═O)O—, —C(═O)N(R^{e3})—, —C(═NR^{e3})—, —C(═NR^{e3})O—, —C(═NR^{e3})N(R^{e3})—, —S(O)₂—, or —S(O)—, and

each instance of R^{A5} is, independently, hydrogen, halogen, optionally substituted aliphatic, optionally substituted heteroaliphatic, optionally substituted aryl, optionally substituted heteroaryl, —CN, —NO₂, —NC, —OR^{A5a}, —SR^{A5b}, —N(R^{A5c})₂, —C(═O)R^{A5d}, —C(═O)OR^{A5a}, —C(═O)N(R^{A5c})₂, —C(═NR^{A5c})R^{A5d}, C(═NR^{A5c})OR^{A5a}, —C(═NR^{A5c})N(R^{A5c})₂, —S(O)₂R^{A5d}, —S(O)R^{A5d}, or two R^{A5} groups adjacent to each other are joined to form a 5- to 6-membered aryl, heteroaryl, heterocyclic or carbocyclic ring, wherein R^{A5a} is hydrogen, an optionally substituted aliphatic, optionally substituted heteroaliphatic, optionally substituted aryl, optionally substituted heteroaryl or a suitable hydroxyl protecting group; wherein R^{A5b} is hydrogen, an optionally substituted aliphatic, optionally substituted heteroaliphatic, optionally substituted aryl, optionally substituted heteroaryl or a suitable thiol protecting group; wherein each R^{A5c} is, independently, hydrogen, an optionally substituted aliphatic, optionally substituted heteroaliphatic, optionally substituted aryl, optionally substituted heteroaryl or a suitable amino protecting group, or two R^{A5c} groups are joined together to form a heterocyclic or heteroaryl group; and wherein each R^{A5d} is, independently, an optionally substituted aliphatic, optionally substituted heteroaliphatic, optionally substituted aryl or an optionally substituted heteroaryl group, and

p is and integer between 0 to 5, inclusive.

In some embodiments, -L- is —C(═O)—.

In some embodiments, -L- is —C(═O)O—.

In some embodiments, -L- is —C(═O)N(R^{e3})—.

In some embodiments, -L- is —C(═NR^{e3})—.

In some embodiments, -L- is —C(═NR^{e3})O—.

In some embodiments, -L- is —C(═NR^{e3})N(R^{e3})—.

In some embodiments, -L- is —S(O)₂—.

In some embodiments, -L- is —S(O)—.

In some embodiments, the group provided by Z, L and R^{L1} is of the formulae:

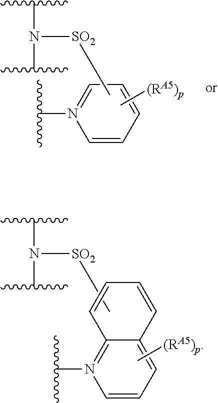

In some embodiments, the group provided by Z, L and R^{L1} is of the formulae:

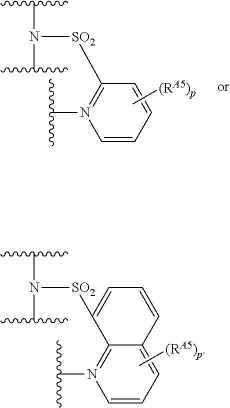

In some embodiments, the group provided by Z, L and R^{L1} is:

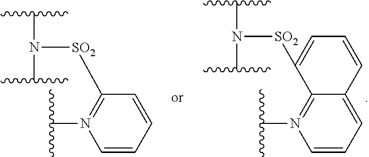

Group Z

In some embodiments, Z is not linked to the ligand R^{L1} as in the case of a palladium (II) complex with a bidentate ligand. As defined generally above, in some embodiments, Z is a bond, —O—, —S—, —C(R^{d})₂—, —C(R^{d})═C(R^{d})—, —C(R^{d})═N—, or —N(R^{e})—;

wherein each instance of R^{d} is, independently, hydrogen, or an optionally substituted aliphatic, optionally substituted heteroaliphatic, optionally substituted aryl, or optionally substituted heteroaryl group; and

each instance of R^{e} is, independently, hydrogen, an optionally substituted aliphatic, optionally substituted heteroaliphatic, optionally substituted aryl, optionally substituted heteroaryl, —C(═O)R^{e1}, —C(═O)OR^{e2}, —C(═O)N(R^{e3})₂, —C(═NR^{e3})R^{e1}, —C(═NR^{e3})OR^{e2}, —C(═NR^{e3})N(R^{e3})₂, —S(O)₂R^{e1}, —S(O)R^{e1}, or a suitable amino protecting group, wherein R^{e1} is an optionally substituted aliphatic, optionally substituted heteroaliphatic, optionally substituted aryl or optionally substituted heteroaryl group; wherein R^{e2} is an optionally substituted aliphatic, optionally substituted heteroaliphatic, optionally substituted aryl, optionally substituted heteroaryl group, or a suitable hydroxyl protecting group; wherein R^{e3} is an optionally substituted aliphatic, optionally substituted heteroaliphatic, optionally substituted aryl, optionally substituted heteroaryl group, or a suitable amino protecting group, or two R^{e3} groups are joined to form an optionally substituted membered heterocyclic or heteroaryl ring.

In some embodiments, Z is a bond.

In some embodiments, Z is —C(R^{d})₂—. In some embodiments, Z is —CH₂—.

In some embodiments, Z is —C(R^{d})═C(R^{d})—. In some embodiments, Z is —CH═CH—.

In some embodiments, Z is —C(R^{d})═N—. In some embodiments, Z is —CH═N—

In some embodiments, Z is —O—.

In some embodiments, Z is —S—.

In some embodiments, Z is —NR^{e}—.

In some embodiments, wherein Z is —NR^{e}—, the R^{e} group is of the formula —S(O)₂R^{e1}, wherein R^{e1} is an optionally substituted aliphatic, optionally substituted heteroaliphatic, optionally substituted aryl or optionally substituted heteroaryl group. In some embodiments, the R^{e} group is of the formula —S(O)₂R^{e1}, wherein R^{e1} is an optionally substituted aryl or optionally substituted heteroaryl group. In some embodiments, the R^{e} group is of the formula —S(O)₂R^{e1}, wherein R^{e1} is an optionally substituted heteroaryl group. In some embodiments, the R^{e} group is of the formula —S(O)₂R^{e1}, wherein R^{e1} is an optionally substituted aryl group.

Exemplary —S(O)₂R^{e1} groups include, but are not limited to:

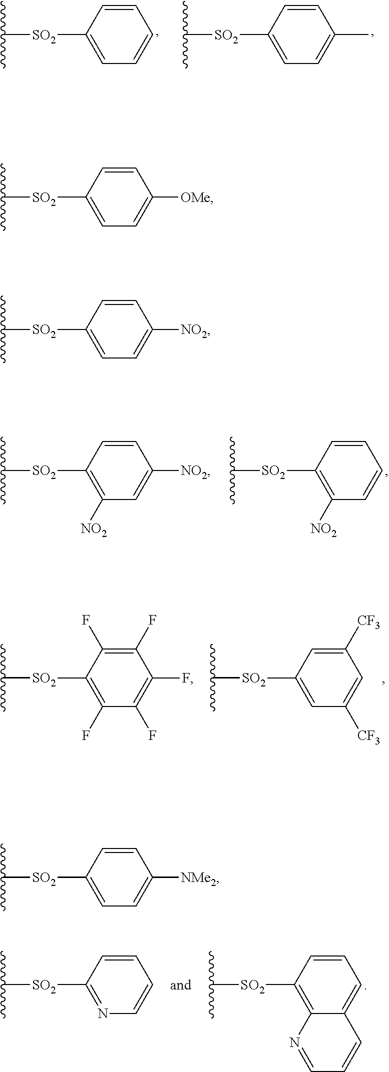

In some embodiments, Z is of the formula:

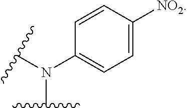

In some embodiments, Z is of the formula:

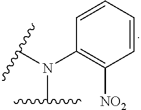

In some embodiments, Z is of the formula:

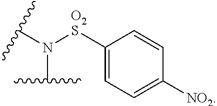

In some embodiments, Z is of the formula:

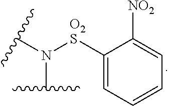

Exemplary Palladium(II) Complexes

In some embodiments, the palladium(II) complex is selected from any of the following complexes:

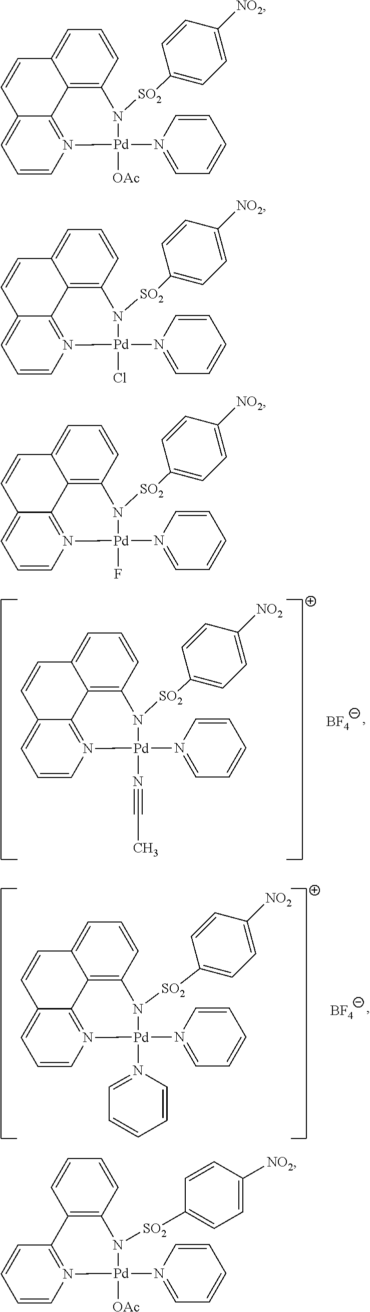

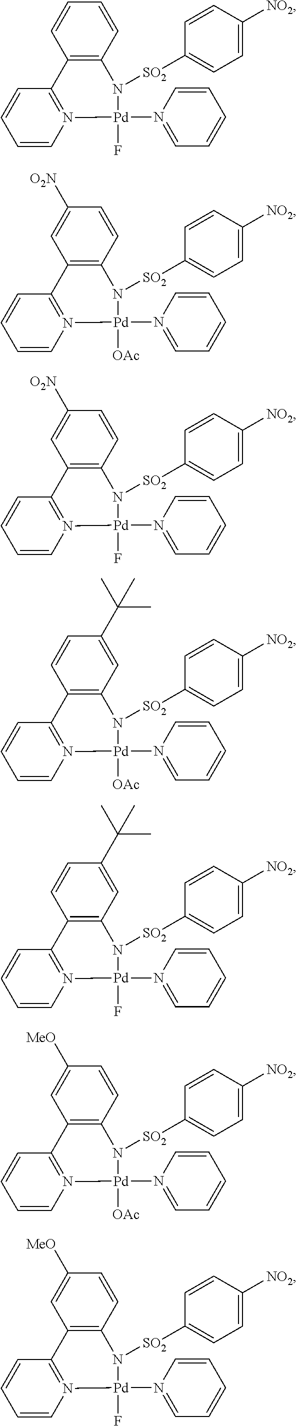

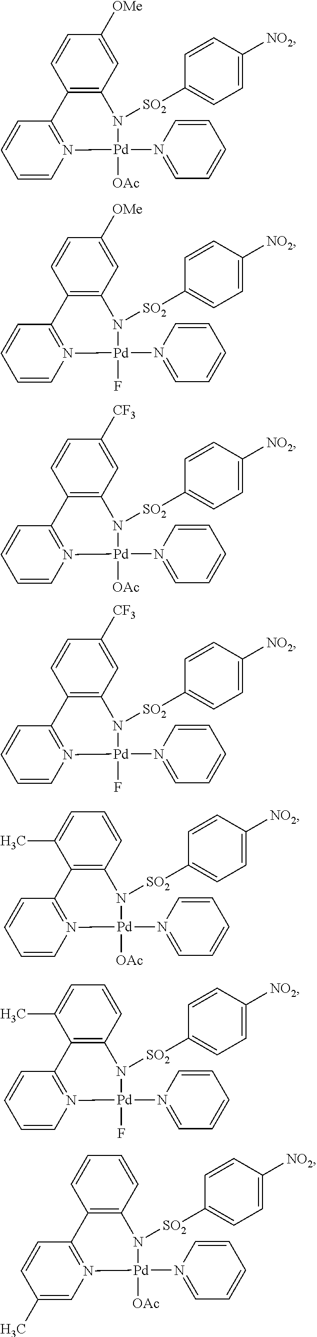

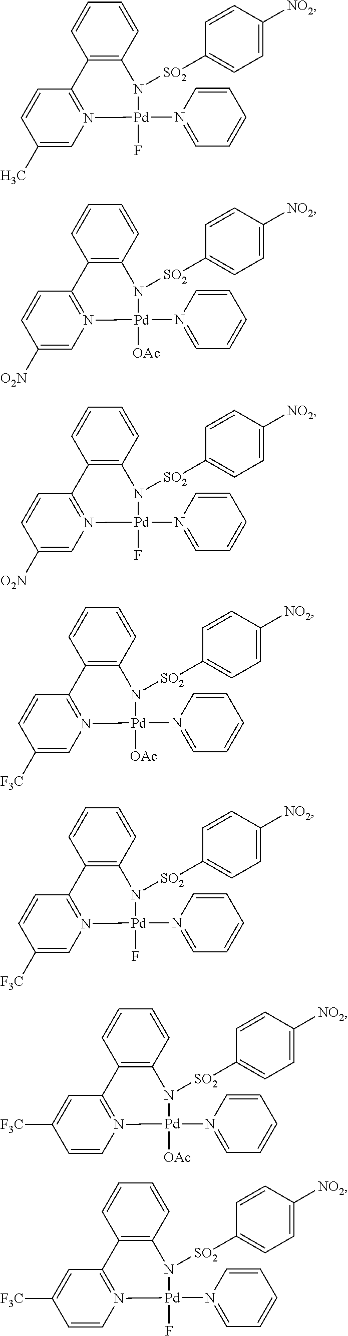

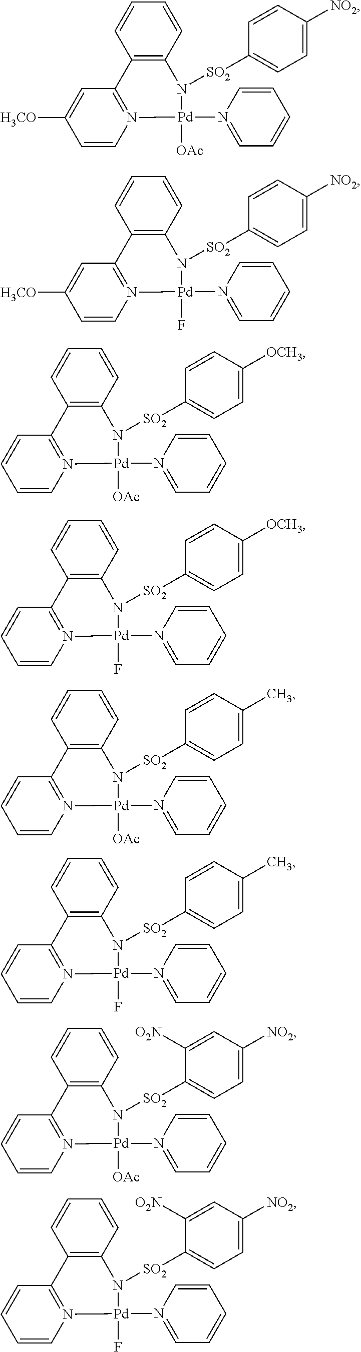

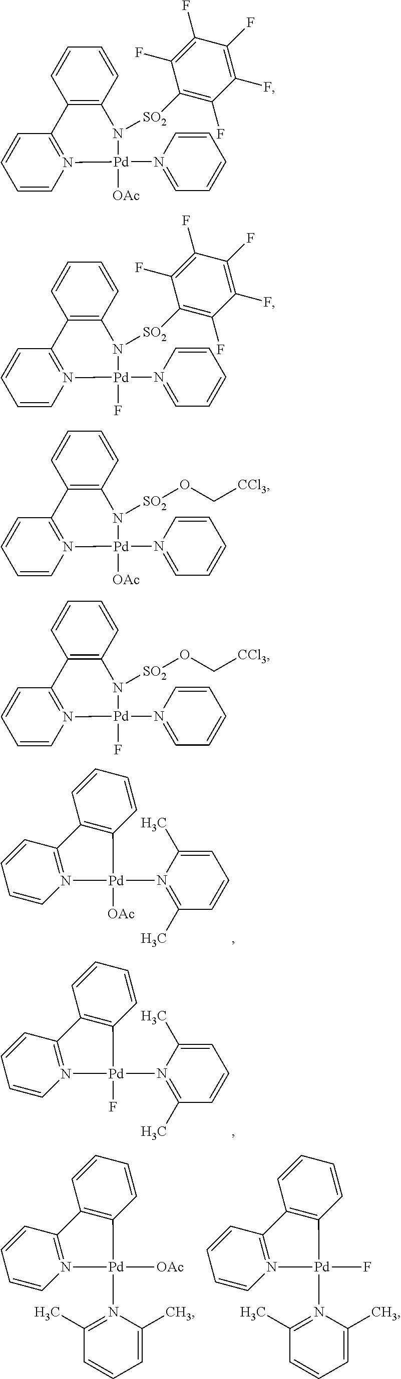

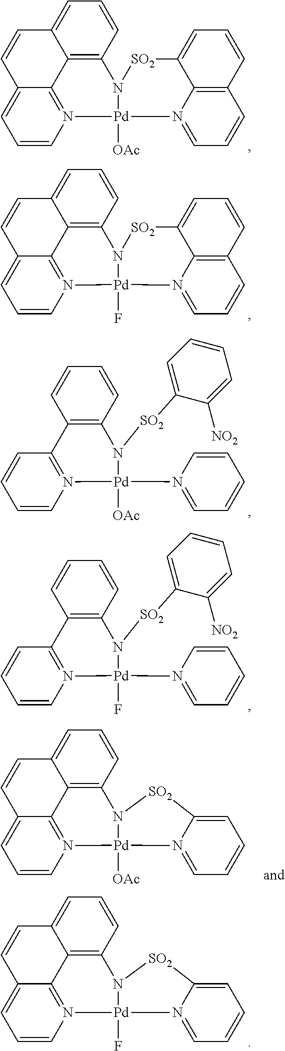

In some embodiments, the palladium (II) complex is of the formula:

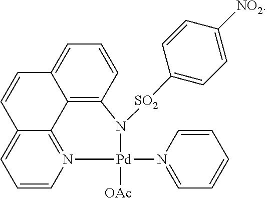

In some embodiments, the palladium(II) complex is of the formula:

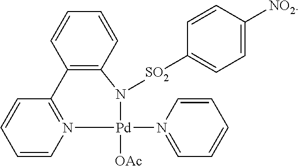

In some embodiments, the palladium(II) complex is of the formula:

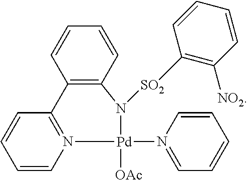

In some embodiments, the palladium(II) complex is of the formula:

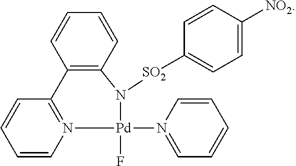

Fluorination with High-Valent Pd(IV)-Fluoride Complexes

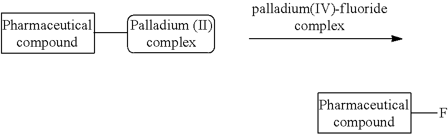

Upon reaction of an organopalladium(II) complex with a high-valent Pd(IV)-fluoride complex, the method provides a fluorinated organic compound in which the organic compound is fluorinated at the position at which it was bound to the palladium(II) center. In some embodiments, the organic compound is attached to the palladium(II) center (and subsequently fluorinated) via an aryl or heteroaryl moiety. For example, see Scheme 8.

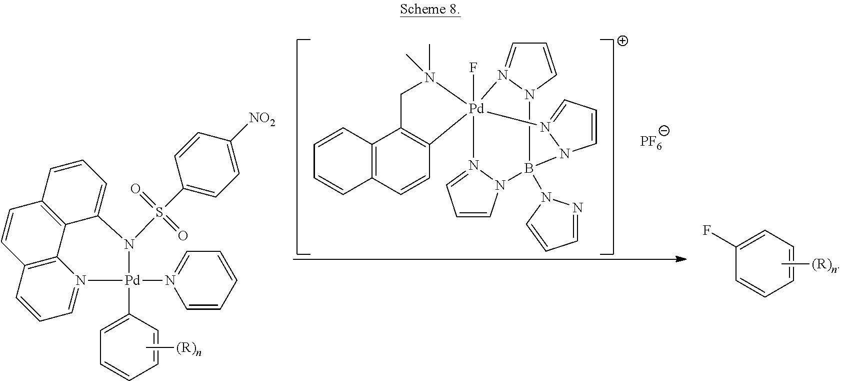

Exemplary methods of fluorinating a compound using a Pd(IV) complex are described in WO2009/149347, which is incorporated herein by reference in its entirety.

Palladium (IV) Complexes

In some embodiments, the complex is a Pd (IV) complex. Typically, the complex comprises one or more bidentate or tridentate ligands. Such ligands, particularly “scorpionate ligands,” are thought to stabilize the octahedral coordination sphere of the palladium (IV) center and thus prevent reductive elimination or other reductive pathways from an octahedral d⁶ palladium (IV) to a square planar d⁸ palladium (II).

In some embodiments, the high-valent palladium fluoride complex is of the formula:

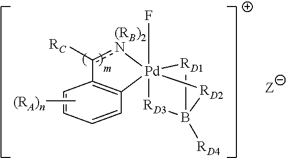

wherein:

the dashed line represents the presence or absence of a bond;

Pd has a valency of +4;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two R_{A} may be taken together to form a substituted or unsubstituted carbocyclic, heterocyclic, aryl or heteroaryl ring;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{C} and R_{B} may be taken together to form a substituted or unsubstituted heterocyclic or heteroaryl ring; and wherein R_{C} and R_{A} may be taken together to form a substituted or unsubstituted carbocyclic, heterocyclic, aryl or heteroaryl ring;

R_{D1}, R_{D2}, R_{D3}, and R_{D4} are each independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl;

Z⁻ is an anion such as halide, acetate, tosylate, azide, tetrafluoroborate, tetraphenylborate, tetrakis(pentafluorophenyl)borate, [B[3,5-(CF₃)₂C₆H₃]₁]⁻, hexafluorophosphate, phosphate, sulfate, perchlorate, trifluoromethanesulfonate or hexafluoroantimonate; and

F comprises ¹⁸F or ¹⁹F.

In some embodiments, the high-valent palladium fluoride complex is of the formula:

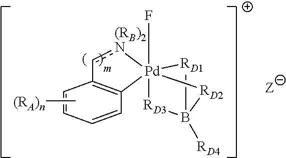

wherein

the dashed line represents the presence or absence of a bond;

Pd has a valency of +4;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two R_{A} may be taken together to form a substituted or unsubstituted carbocyclic, heterocyclic, aryl or heteroaryl ring;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

R_{D1}, R_{D2}, R_{D3}, and R_{D4} are each independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl;

Z⁻ is an anion such as halide, acetate, tosylate, azide, tetrafluoroborate, tetraphenylborate, tetrakis(pentafluorophenyl)borate, [B[3,5-(CF₃)₂C₆H₃]₄]⁻, hexafluorophosphate, phosphate, sulfate, perchlorate, trifluoromethanesulfonate or hexafluoroantimonate; and

F comprises ¹⁸F or ¹⁹F.

The counteranion Z⁻ may be any suitable anion. In some embodiments, the counteranion has a charge of −1. In some embodiments, the counteranion has a charge of −2. In some embodiments, the counteranion has a charge of −3. The counteranion may be an organic or inorganic anion. In some embodiments, the counteranion is an inorganic anion such as phosphate, hexafluorophosphate, hexafluoroantimonate, sulfate, perchlorate, azide, a halide such as fluoride, chloride, bromide or iodide, etc. In other embodiments, the counteranion is an organic anion such as a carboxylate (e.g., acetate), sulfonate, phosphonate, borate, etc. In some embodiments, the counteranion is trifluoromethanesulfonate (triflate). In some embodiments, the counteranion is tosylate. In some embodiments, the counteranion is mesylate. In some embodiments, the counteranion is hexafluorophosphate. In some embodiments, the counteranion is tetraphenylborate. In some embodiments, the counteranion is tetrafluoroborate. In some embodiments, the counteranion tetrakis(pentafluorophenyl)borate. In some embodiments, the counteranion is hexafluoroantimonate. In some embodiments, the counterion is [B[3,5-(CF₃)₂C₆H₃]₄]⁻, commonly abbreviated as [BAr^{F}₄]⁻.

In some embodiments, n is 0, in which case the phenyl ring is unsubstituted. In some embodiments, n is 1. In some embodiments, n is 2. In some embodiments, n is 3. In some embodiments, n is 4. For the case where n is 1 or more, the substituents on the phenyl ring may have any substitution pattern.

In some embodiments, m is 0. In some embodiments, m is 1. In some embodiments, m is 2. In some embodiments, m is 3.

In some embodiments, the dashed line represents a bond, thus forming an imine moiety. In other embodiments, the dashed line represents the absence of a bond resulting in only a single bond between the carbon atom and nitrogen atom.

In some embodiments, at least one R_{A} is halogen. In some embodiments, at least one occurrence of R_{A} is aliphatic. In some embodiments, at least one occurrence of R_{A} is C₁-C₆ alkyl. In some embodiments, at least one occurrence of R_{A} is methyl. In some embodiments, at least one occurrence of R_{A} is ethyl. In some embodiments, at least one occurrence of R_{A} is propyl. In some embodiments, at least one occurrence of R_{A} is butyl. In some embodiments, at least one occurrence of R_{A} is heteroaliphatic. In some embodiments, at least one occurrence of R_{A} is acyl. In some embodiments, at least one occurrence of R_{A} is aryl. In some embodiments, at least one occurrence of R_{A} is heteroaryl. In some embodiments, at least one occurrence of R_{A} is —OR′. In some embodiments, at least one occurrence of R_{A} is —N(R′)₂. In some embodiments, at least one occurrence of R_{A} is —SR′. In some embodiments, at least one occurrence of R_{A} is —NO₂. In some embodiments, at least one occurrence of R_{A} is —CN. In some embodiments, at least one occurrence of R_{A} is —SCN.

In some embodiments, two occurrences of R_{A} taken together form a cyclic moiety. Such a cyclic moiety may be carbocyclic or heterocyclic. In some embodiments, the cyclic moiety is a substituted or unsubstituted phenyl moiety. In some embodiments, the cyclic moiety is an unsubstituted phenyl moiety. In some embodiments, the cyclic moiety is a substituted or unsubstituted heteroaryl moiety.

In some embodiments, at least one occurrence of R_{B} is hydrogen. In some embodiments, both R_{B} are hydrogen. In some embodiments, at least one occurrence of R_{B} is aliphatic. In some embodiments, both occurrences of R_{B} are aliphatic. In some embodiments, both occurrences of R_{B} are C₁-C₆ alkyl. In some embodiments, both occurrences of R_{B} are methyl. In some embodiments, both occurrences of R_{B} are ethyl. In some embodiments, both occurrences of R_{B} are propyl. In some embodiments, both occurrences of R_{B} are butyl. In some embodiments, at least one occurrence of R_{B} is heteroaliphatic. In some embodiments, both occurrences of R_{B} are heteroaliphatic. In some embodiments, at least one occurrence of R_{B} is acyl. In some embodiments, at least one occurrence of R_{B} is aryl. In some embodiments, at least one occurrence of R_{B} is heteroaryl.

In some embodiments, both R_{B} are the same. In some embodiments, the two R_{B} are different.

In some embodiments, both R_{B} are taken together to form a heterocyclic moiety. In some embodiments, both R_{B} are taken together to form a 5-membered heterocyclic moiety. In some embodiments, both R_{B} are taken together to form a 6-membered heterocyclic moiety. In some embodiments, both R_{B} are taken together to form an optionally substituted heteroaryl moiety.

In some embodiments, one R_{B} moiety is covalently attached to a methylene group connecting the phenyl ring to the N atom, thus forming a heterocyclic moiety. Such a heterocyclic moiety may be a heteroaryl moiety. For example, in some embodiments, the heterocyclic moiety is a pyridinyl moiety.

In some embodiments, R_{C} is hydrogen. In some embodiments, R_{C} is aliphatic. In some embodiments, R_{C} is C₁-C₆ alkyl. In some embodiments, R_{C} is methyl. In some embodiments, R_{C} is ethyl. In some embodiments, R_{C} is propyl. In some embodiments, R_{C} is butyl. In some embodiments, R_{C} is heteroaliphatic. In some embodiments, R_{C} is heteroaliphatic. In some embodiments, R_{C} is acyl. In some embodiments, R_{C} is aryl. In some embodiments, R_{C} is heteroaryl. In some embodiments, one R_{B} and R_{C} are taken together to form a heterocyclic moiety. In some embodiments, one R_{B} and R_{C} are taken together to form a 5-membered heterocyclic moiety. In some embodiments, one R_{B} and R_{C} are taken together to form a 6-membered heterocyclic moiety. In some embodiments, one R_{B} and R_{C} are taken together to form an optionally substituted heteroaryl moiety.

In some embodiments, R_{D1}, R_{D2}, R_{D3} and R_{D4} all represent optionally substituted heteroaryl moieties. In some embodiments, at least one of R_{D1}, R_{D2}, R_{D3} and R_{D4} is an unsubstituted heteroaryl moiety. In some embodiments, R_{D1}, R_{D2}, R_{D3} and R_{D4} are all unsubstituted heteroaryl moieties. In some embodiments, R_{D1}, R_{D2}, R_{D3} and R_{D4} are all optionally substituted 5-membered heteroaryl moieties. In some embodiments, R_{D1}, R_{D2}, R_{D3} and R_{D4} are all nitrogen-containing 5-membered heteroaryl moieties, which are optionally substituted. In some embodiments, R_{D1}, R_{D2}, R_{D3} and R_{D4} are all optionally substituted pyrazolyl moieties. In some embodiments, R_{D1}, R_{D2}, R_{D3} and R_{D4} are all optionally substituted imidazolyl moieties. In some embodiments, R_{D1}, R_{D2}, R_{D3} and R_{D4} are all optionally substituted pyrrolyl moieties. In some embodiments, R_{D1}, R_{D2}, R_{D3} and R_{D4} are all are optionally substituted thiazolyl moieties. In some embodiments, R_{D1}, R_{D2}, R_{D3} and R_{D4} are all optionally substituted oxazolyl moieties. In some embodiments, R_{D1}, R_{D2}, R_{D3} and R_{D4} are all optionally substituted 6-membered heteroaryl moieties. In some embodiments, R_{D1}, R_{D2}, R_{D3} and R_{D4} are all nitrogen-containing 6-membered heteroaryl moieties, which are optionally substituted. In some embodiments, R_{D1}, R_{D2}, R_{D3} and R_{D4} are all optionally substituted pyridinyl moieties. In some embodiments, R_{D1}, R_{D2}, R_{D3} and R_{D4} are all optionally substituted pyrazinyl moieties. In some embodiments, R_{D1}, R_{D2}, R_{D3} and R_{D4} are all optionally substituted pyrimidinyl moieties. In some embodiments, R_{D1}, R_{D2}, R_{D3} and R_{D4} are all optionally substituted pyridazinyl moieties. In some embodiments, all of R_{D1}, R_{D2}, R_{D3} and R_{D4} of the borate ligand are the same. In other embodiments, all of R_{D1}, R_{D2}, R_{D3} and R_{D4} of the borate ligand are not the same. For example, a combination of heterocycle may constitute the borate ligand. In some embodiments, a combination of heteroaryl moieties may constitute the borate ligand.

In some embodiments, the palladium complex comprises a bidentate ligand of one of the formulae:

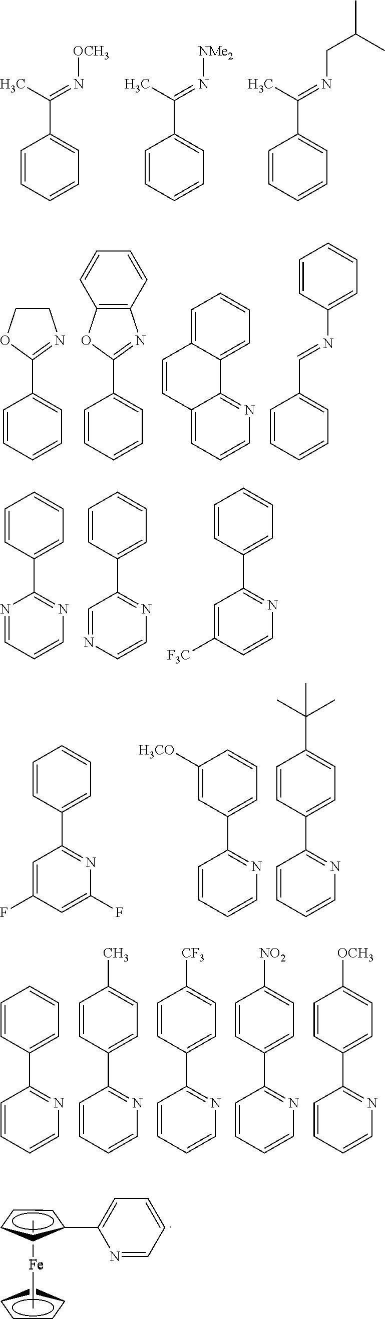

These ligands make a five-membered ring with the palladium atom with the nitrogen and a carbon coordinated to the central palladium.

In some embodiments, the palladium complex is of the formula:

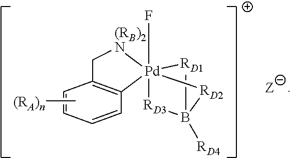

In some embodiments, the palladium complex is of the formula:

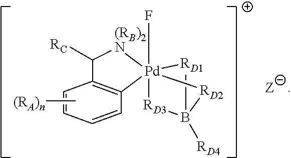

In some embodiments, the palladium complex is of the formula:

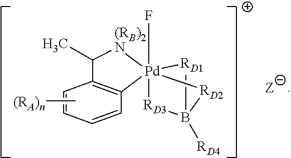

In some embodiments, the palladium complex is of the formula:

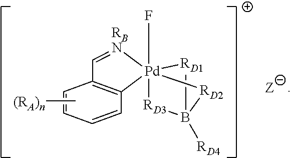

In some embodiments, the palladium complex is of the formula:

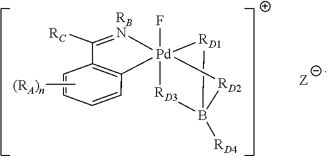

In some embodiments, the palladium complex is of the formula:

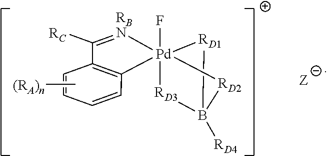

In some embodiments, the palladium complex is of the formula:

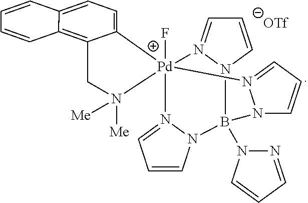

In some embodiments, the palladium complex is of the formula:

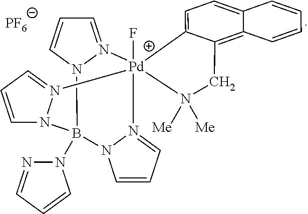

Preparation of High-Valent Palladium Fluoride Complexes

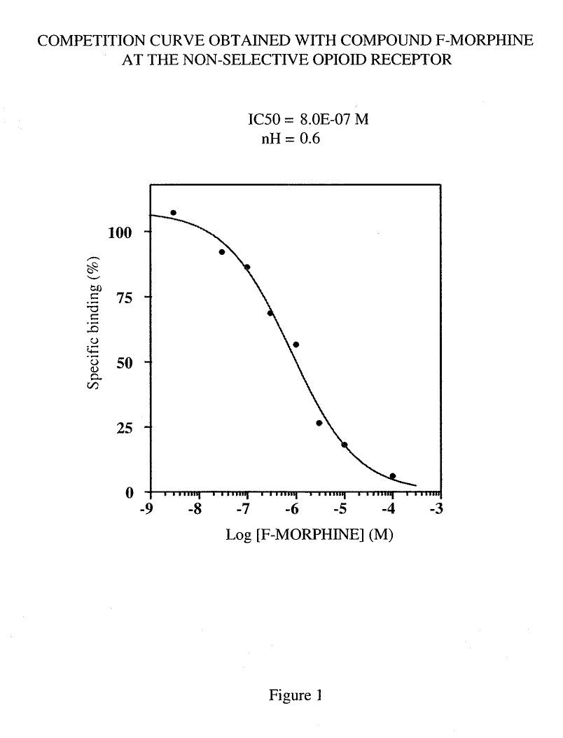

The palladium complexes are typically prepared starting from disodium tetrachloropalladate. As would be appreciated by one of skill in the art, other palladium salts may also be used to prepare the complexes. The starting material is subjected to cyclometallation to yield a palladium(II) chloride dimer. The chloride ligands are then substituted using the desired borate ligand to yield a palladium(II) borate, which is then oxidized with a fluorine-containing oxidizing reagent (e.g., 1-fluoro-pyridinium triflate, 2,4,6-trimethylpyridinium hexafluorophosphate, etc.) to yield the palladium(IV) complex. An exemplary synthesis of a palladium(IV) fluoride complex is shown in FIG. 1.

In some embodiments, the method of preparing an palladium(IV) fluoride complex comprises (1) cyclometallating a palladium(II) salt with a bidentate ligand comprising a carbon-based with a carbon donor and a nitrogen donor to yield a palladium(II) chloride dimer; (2) reacting the palladium(II) dimer with a tridentate borate ligand under suitable conditions to yield a palladium(II) borate; and oxidizing the palladium(II) borate with a fluorinating reagent under suitable conditions to yield a palladium(IV) fluoride complex.

In some embodiments, the bidentate ligand is of the formula:

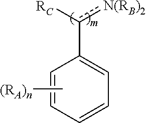

wherein

the dashed line represents the presence or absence of a bond;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two RA may be taken together to form a substituted or unsubstituted carbocyclic, heterocyclic, aryl or heteroaryl ring; and

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; and

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{C} and R_{B} may be taken together to form a substituted or unsubstituted heterocyclic or heteroaryl ring; and wherein R_{C} and R_{A} may be taken together to form a substituted or unsubstituted carbocyclic, heterocyclic, aryl or heteroaryl ring.

In some embodiments, the borate ligand is tetrapyrazolylborate. In some embodiments, the borate ligand is phenyltris(methimazolyl)borate.

In some embodiments, an intermediate in the synthesis of a palladium(IV) fluoride complex is of the formula:

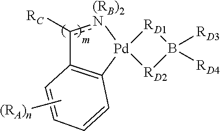

wherein

the dashed line represents the presence or absence of a bond;

Pd has a valency of +2;

n is an integer between 0 and 4, inclusive;

m is an integer between 0 and 3, inclusive;

each occurrence of R_{A} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR′; —C(═O)R′; —CO₂R′; —CN; —SCN; —SR′; —SOR′; —SO₂R′; —NO₂; —N(R′)₂; —NHC(O)R′; or —C(R′)₃; wherein each occurrence of R′ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety; wherein two R_{A} may be taken together to form a substituted or unsubstituted carbocyclic, heterocyclic, aryl or heteroaryl ring;

each occurrence of R_{B} is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OR″; —C(═O)R″; —CO₂R″; —CN; —SCN; —SR″; —SOR″; —SO₂R″; —NO₂; —N(R″)₂; —NHC(O)R″; or —C(R″)₃; wherein each occurrence of R″ is independently a hydrogen, a protecting group, an aliphatic moiety, a heteroaliphatic moiety, an acyl moiety; an aryl moiety; a heteroaryl moiety; alkoxy; aryloxy; alkylthio; arylthio; amino, alkylamino, dialkylamino, heteroaryloxy; or heteroarylthio moiety;

each occurrence of R_{C} is independently hydrogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; wherein R_{C} and R_{B} may be taken together to form a substituted or unsubstituted heterocyclic or heteroaryl ring; and wherein R_{C} and R_{A} may be taken together to form a substituted or unsubstituted carbocyclic, heterocyclic, aryl or heteroaryl ring; and

R_{D1}, R_{D2}, R_{D3}, and R_{D4} are each independently cyclic or acyclic, substituted or unsubstituted, branched or unbranched aliphatic; cyclic or acyclic, substituted or unsubstituted, branched or unbranched heteroaliphatic; substituted or unsubstituted, branched or unbranched aryl; substituted or unsubstituted, branched or unbranched heteroaryl.

In some embodiments, n is 0, in which case the phenyl ring is unsubstituted. In some embodiments, n is 1. In some embodiments, n is 2. In some embodiments, n is 3. In some embodiments, n is 4. For the case where n is 1 or more, the substituents on the phenyl ring may have any substitution pattern.

In some embodiments, m is 0. In some embodiments, m is 1. In some embodiments, m is 2. In some embodiments, m is 3.

In some embodiments, the dashed line represents a bond, thus forming an imine moiety. In other embodiments, the dashed line represents the absence of a bond resulting in only a single bond between the carbon atom and nitrogen atom.

In some embodiments, at least one R_{A} is halogen. In some embodiments, at least one occurrence of R_{A} is aliphatic. In some embodiments, at least one occurrence of R_{A} is C₁-C₆ alkyl. In some embodiments, at least one occurrence of R_{A} is methyl. In some embodiments, at least one occurrence of R_{A} is ethyl. In some embodiments, at least one occurrence of R_{A} is propyl. In some embodiments, at least one occurrence of R_{A} is butyl. In some embodiments, at least one occurrence of R_{A} is heteroaliphatic. In some embodiments, at least one occurrence of R_{A} is acyl. In some embodiments, at least one occurrence of R_{A} is aryl. In some embodiments, at least one occurrence of R_{A} is heteroaryl. In some embodiments, at least one occurrence of R_{A} is —OR′. In some embodiments, at least one occurrence of R_{A} is —N(R′)₂. In some embodiments, at least one occurrence of R_{A} is —SR′. In some embodiments, at least one occurrence of R_{A} is —NO₂. In some embodiments, at least one occurrence of R_{A} is —CN. In some embodiments, at least one occurrence of R_{A} is —SCN.

In some embodiments, two occurrences of R_{A} taken together form a cyclic moiety. Such a cyclic moiety may be carbocyclic or heterocyclic. In some embodiments, the cyclic moiety is a substituted or unsubstituted phenyl moiety. In some embodiments, the cyclic moiety is an unsubstituted phenyl moiety. In some embodiments, the cyclic moiety is a substituted or unsubstituted heteroaryl moiety.

In some embodiments, at least one occurrence of R_{B} is hydrogen. In some embodiments, both R_{B} are hydrogen. In some embodiments, at least one occurrence of R_{B} is aliphatic. In some embodiments, both occurrences of R_{B} are aliphatic. In some embodiments, both occurrences of R_{B} are C₁-C₆ alkyl. In some embodiments, both occurrences of R_{B} are methyl. In some embodiments, both occurrences of R_{B} are ethyl. In some embodiments, both occurrences of R_{B} are propyl. In some embodiments, both occurrences of R_{B} are butyl. In some embodiments, at least one occurrence of R_{B} is heteroaliphatic. In some embodiments, both occurrences of R_{B} are heteroaliphatic. In some embodiments, at least one occurrence of R_{B} is acyl. In some embodiments, at least one occurrence of R_{B} is aryl. In some embodiments, at least one occurrence of R_{B} is heteroaryl.

In some embodiments, both R_{B} are the same. In some embodiments, the two R_{B} are different.

In some embodiments, both R_{B} are taken together to form a heterocyclic moiety. In some embodiments, both R_{B} are taken together to form a 5-membered heterocyclic moiety. In some embodiments, both R_{B} are taken together to form a 6-membered heterocyclic moiety. In some embodiments, both R_{B} are taken together to form an optionally substituted heteroaryl moiety.

In some embodiments, one R_{B} moiety is covalently attached to a methylene group connecting the phenyl ring to the N atom, thus forming a heterocyclic moiety. Such a heterocyclic moiety may be a heteroaryl moiety. For example, in some embodiments, the heterocyclic moiety is a pyridinyl moiety.

In some embodiments, R_{D1}, R_{D2}, R_{D3} and R_{D4} all represent optionally substituted heteroaryl moieties. In some embodiments, at least one of R_{D1}, R_{D2}, R_{D3} and R_{D4} is an unsubstituted heteroaryl moiety. In some embodiments, R_{D1}, R_{D2}, R_{D3} and R_{D4} are all unsubstituted heteroaryl moieties. In some embodiments, R_{D1}, R_{D2}, R_{D3} and R_{D4} are all optionally substituted 5-membered heteroaryl moieties. In some embodiments, R_{D1}, R_{D2}, R_{D3} and R_{D4} are all nitrogen-containing 5-membered heteroaryl moieties, which are optionally substituted. In some embodiments, R_{D1}, R_{D2}, R_{D3} and R_{D4} are all optionally substituted pyrazolyl moieties. In some embodiments, R_{D1}, R_{D2}, R_{D3} and R_{D4} are all optionally substituted imidazolyl moieties. In some embodiments, R_{D1}, R_{D2}, R_{D3} and R_{D4} are all optionally substituted pyrrolyl moieties. In some embodiments, R_{D1}, R_{D2}, R_{D3} and R_{D4} are all are optionally substituted thiazolyl moieties. In some embodiments, R_{D1}, R_{D2}, R_{D3} and R_{D4} are all optionally substituted oxazolyl moieties. In some embodiments, R_{D1}, R_{D2}, R_{D3} and R_{D4} are all optionally substituted 6-membered heteroaryl moieties. In some embodiments, R_{D1}, R_{D2}, R_{D3} and R_{D4} are all nitrogen-containing 6-membered heteroaryl moieties, which are optionally substituted. In some embodiments, R_{D1}, R_{D2}, R_{D3} and R_{D4} are all optionally substituted pyridinyl moieties. In some embodiments, R_{D1}, R_{D2}, R_{D3} and R_{D4} are all optionally substituted pyrazinyl moieties. In some embodiments, R_{D1}, R_{D2}, R_{D3} and R_{D4} are all optionally substituted pyrimidinyl moieties. In some embodiments, R_{D1}, R_{D2}, R_{D3} and R_{D4} are all optionally substituted pyridazinyl moieties. In some embodiments, all of R_{D1}, R_{D2}, R_{D3} and R_{D4} of the borate ligand are the same. In other embodiments, all of R_{D1}, R_{D2}, R_{D3} and R_{D4} of the borate ligand are not the same. For example, a combination of heterocycle may constitute the borate ligand. In some embodiments, a combination of heteroaryl moieties may constitute the borate ligand.

In some embodiments, the intermediate comprises a bidentate ligand of one of the formulae:

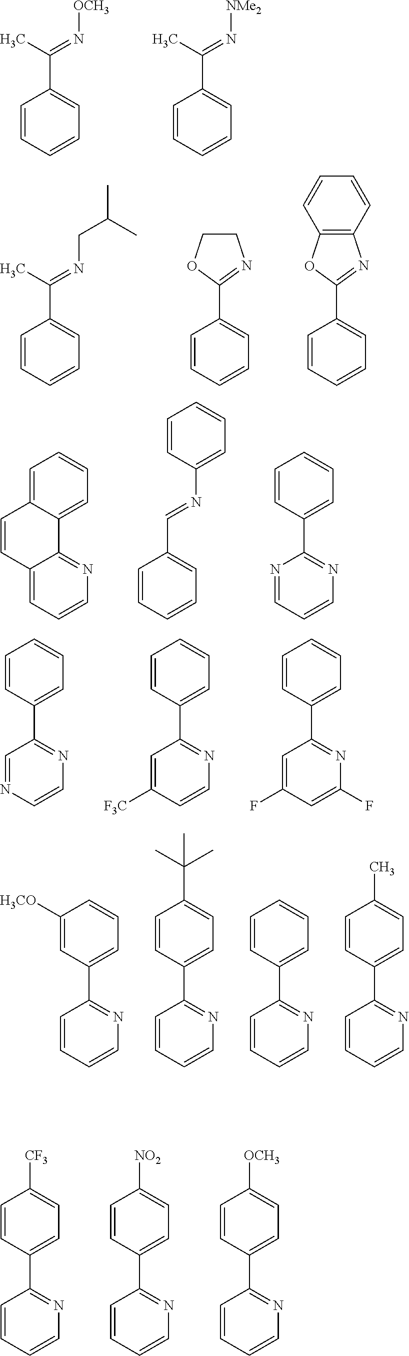

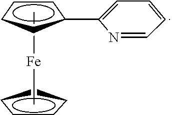

These ligands make a five-membered ring with the palladium atom with the nitrogen and a carbon coordinated to the central palladium.

In some embodiments, the intermediate is of the formula:

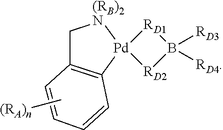

In some embodiments, the intermediate is of the formula:

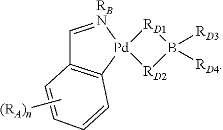

In some embodiments, the intermediate is of the formula:

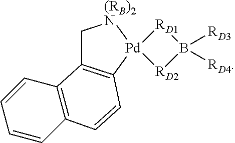

In some embodiments, the intermediate is of the formula:

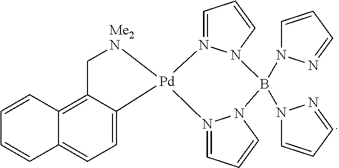

As can be appreciated by the skilled artisan, alternative methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

Methods of Treatment

The compounds and compositions described herein can be administered to cells in culture, e.g. in vitro or ex vivo, or to a subject, e.g., in vivo, to treat, prevent, and/or diagnose a variety of disorders, including those described herein below.

As used herein, the term “treat” or “treatment” is defined as the application or administration of a compound, alone or in combination with, a second compound to a subject, e.g., a patient, or application or administration of the compound to an isolated tissue or cell, e.g., cell line, from a subject, e.g., a patient, who has a disorder (e.g., a disorder as described herein), a symptom of a disorder, or a predisposition toward a disorder, with the purpose to cure, heal, alleviate, relieve, alter, remedy, ameliorate, improve or affect the disorder, one or more symptoms of the disorder or the predisposition toward the disorder (e.g., to prevent at least one symptom of the disorder or to delay onset of at least one symptom of the disorder).

As used herein, an amount of a compound effective to treat a disorder, or a “therapeutically effective amount” refers to an amount of the compound which is effective, upon single or multiple dose administration to a subject, in treating a cell, or in curing, alleviating, relieving or improving a subject with a disorder beyond that expected in the absence of such treatment.

As used herein, an amount of a compound effective to prevent a disorder, or a “a prophylactically effective amount” of the compound refers to an amount effective, upon single- or multiple-dose administration to the subject, in preventing or delaying the occurrence of the onset or recurrence of a disorder or a symptom of the disorder.

As used herein, the term “subject” is intended to include human and non-human animals. Exemplary human subjects include a human patient having a disorder, e.g., a disorder described herein or a normal subject. The term “non-human animals” of the invention includes all vertebrates, e.g., non-mammals (such as chickens, amphibians, reptiles) and mammals, such as non-human primates, domesticated and/or agriculturally useful animals, e.g., sheep, dog, cat, cow, pig, etc.

Described herein are compounds and compositions useful as opioid analgesics, and also compounds used to treat opioid dependence, such as an opioid analgesic or opioid dependence agent described herein. In general, the compounds described herein are fluorinated derivatives of a pharmaceutical agent (e.g., an opioid receptor agonist). Also envisioned herein are other opioid analgesics and agents for treating opioid dependence, wherein one or more fluorine moieties have been added to the pharmaceutical agent, e.g., replacing a hydrogen or functional group such as an —OH with a fluorine.

Opioids

An opioid is a chemical substance that has a morphine-like action in the body. There are a number of broad classes of opioids, including, natural opiates (alkaloids contained in the resin of the opium poppy including morphine, codeine and thebaine), semi-synthetic opiates (created from the natural opioids), fully synthetic opioids, and endogenous opioid peptides (produced naturally in the body).

Opioids can be used for pain relief. These agents generally work by binding to opioid receptors, which are found principally in the central nervous system and the gastrointestinal tract. There are three principal classes of opioid receptors, μ, κ, δ, although up to seventeen have been reported, and include the ε, τ, λ, and ζ receptors. In addition, there are three subtypes of μ receptor: μ1 and μ2, and the newly discovered μ3. Another receptor of clinical importance is the opioid-receptor-like receptor 1 (ORL1), which is involved in pain responses as well as having a major role in the development of tolerance to μ-opioid agonists used as analgesics. These are all G-protein coupled receptors acting on GABAergic neurotransmission. The pharmacodynamic response to an opioid depends on which receptor it binds, its affinity for that receptor, and whether the opioid is an agonist or an antagonist. For example, the supraspinal analgesic properties of the opioid agonist morphine are mediated by activation of the μ1 receptor, respiratory depression and physical dependence (dependency) by the μ2 receptor, and sedation and spinal analgesia by the κ receptor. Each group of opioid receptors elicits a distinct set of neurological responses, with the receptor subtypes (such as μ1 and μ2 for example) providing even more specific responses. Unique to each opioid is their distinct binding affinity to the group(s) of opioid receptors (e.g., the μ, κ, and δ opioid receptors are activated at different magnitudes according to the specific receptor binding affinities of the opioid, such as the μ opioid receptor effects being the primary receptor response to the opioid morphine, or the κ opioid receptor residing as the primary binding receptor to ketazocine).

Clinical use of opioids include, e.g., Analgesia i.e. to combat pain of various types and induction and the continuance of anesthesia as well as allaying patient apprehension right before the procedure (Fentanyl, oxymorphone, hydromorphone, and morphine are commonly used for this purpose), Cough (codeine, dihydrocodeine, ethylmorphine (dionine), hydromorphone and hydrocodone, with morphine or methadone can be used for this purpose), Diarrhoea (generally loperamide, difenoxin or diphenoxylate, but paregoric, powdered opium or laudanum or morphine may be used in some cases of severe diarrheal diseases), Diarrhoea of Irritable Bowel Syndrome (e.g., Codeine, paregoric, diphenoxylate, difenoxin, loperamide, laudanum), Anxiety due to shortness of breath (e.g., oxymorphone and dihydrocodeine), and Detoxification (e.g., methadone and buprenorphine).

In some instances, where a subject has become dependent on an opioid, the subject is administered a compound to treat the opioid dependence. Opioid dependency is a medical diagnosis characterized by an individual's inability to stop using opioids even when objectively in his or her best interest to do so. In 1964 the WHO Expert Committee on Drug Dependence introduced “dependence” as “A cluster of physiological, behavioral and cognitive phenomena of variable intensity, in which the use of a psychoactive drug (or drugs) takes on a high priority. The necessary descriptive characteristics are preoccupation with a desire to obtain and take the drug and persistent drug-seeking behaviour. Treatment approaches include abstinence-based and harm-reduction methodologies. Both include participation in detoxification through the use of methadone or other long-acting opioids. Alternative detox protocols call for total abstention from all opiates, with the use of various benzodiazepines and other medications to reduce the uncomfortable withdrawal symptoms associated with abstinence. In an abstinence-based approach, a gradual taper of the medications follows detox, while in the harm-reduction approach, the patient remains on an ongoing dose of methadone or buprenorphine.

Compositions and Routes of Administration

The compositions delineated herein include the compounds delineated herein (e.g., a compound described herein), as well as additional therapeutic agents if present, in amounts effective for achieving a modulation of disease or disease symptoms, including those described herein.

The term “pharmaceutically acceptable carrier or adjuvant” refers to a carrier or adjuvant that may be administered to a patient, together with a compound of this invention, and which does not destroy the pharmacological activity thereof and is nontoxic when administered in doses sufficient to deliver a therapeutic amount of the compound.

Pharmaceutically acceptable carriers, adjuvants and vehicles that may be used in the pharmaceutical compositions of this invention include, but are not limited to, ion exchangers, alumina, aluminum stearate, lecithin, self-emulsifying drug delivery systems (SEDDS) such as d-α-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as α-, β-, and γ-cyclodextrin, or chemically modified derivatives such as hydroxyalkylcyclodextrins, including 2- and 3-hydroxypropyl-β-cyclodextrins, or other solubilized derivatives may also be advantageously used to enhance delivery of compounds of the formulae described herein.

The pharmaceutical compositions of this invention may be administered orally, parenterally, by inhalation spray, topically, rectally, nasally, buccally, vaginally or via an implanted reservoir, preferably by oral administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases or buffers to enhance the stability of the formulated compound or its delivery form. The term parenteral as used herein includes subcutaneous, intracutaneous, intravenous, intramuscular, intraarticular, intraarterial, intrasynovial, intrasternal, intrathecal, intralesional and intracranial injection or infusion techniques.

The pharmaceutical compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

Topical administration of the pharmaceutical compositions of this invention is useful when the desired treatment involves areas or organs readily accessible by topical application. For application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax and water. Alternatively, the pharmaceutical composition can be formulated with a suitable lotion or cream containing the active compound suspended or dissolved in a carrier with suitable emulsifying agents. Suitable carriers include, but are not limited to, mineral oil, sorbitan monostearate, polysorbate 60, cetyl esters wax, cetearyl alcohol, 2-octyldodecanol, benzyl alcohol and water. The pharmaceutical compositions of this invention may also be topically applied to the lower intestinal tract by rectal suppository formulation or in a suitable enema formulation. Topically-transdermal patches are also included in this invention.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

The compounds described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation, with a dosage ranging from about 0.5 to about 100 mg/kg of body weight, alternatively dosages between 1 mg and 1000 mg/dose, every 4 to 120 hours, or according to the requirements of the particular drug. The methods herein contemplate administration of an effective amount of compound or compound composition to achieve the desired or stated effect. Typically, the pharmaceutical compositions of this invention will be administered from about 1 to about 6 times per day or alternatively, as a continuous infusion. Such administration can be used as a chronic or acute therapy. The amount of active ingredient that may be combined with the carrier materials to produce a single dosage form will vary depending upon the host treated and the particular mode of administration. A typical preparation will contain from about 5% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, and the judgment of the treating physician.

Upon improvement of a patient's condition, a maintenance dose of a compound, composition or combination of this invention may be administered, if necessary. Subsequently, the dosage or frequency of administration, or both, may be reduced, as a function of the symptoms, to a level at which the improved condition is retained when the symptoms have been alleviated to the desired level. Patients may, however, require intermittent treatment on a long-term basis upon any recurrence of disease symptoms.

Kits

A compound described herein described herein can be provided in a kit. The kit includes (a) a compound described herein, e.g., a composition that includes a compound described herein, and, optionally (b) informational material. The informational material can be descriptive, instructional, marketing or other material that relates to the methods described herein and/or the use of a compound described herein for the methods described herein.

The informational material of the kits is not limited in its form. In one embodiment, the informational material can include information about production of the compound, molecular weight of the compound, concentration, date of expiration, batch or production site information, and so forth. In one embodiment, the informational material relates to methods for administering the compound.

In one embodiment, the informational material can include instructions to administer a compound described herein in a suitable manner to perform the methods described herein, e.g., in a suitable dose, dosage form, or mode of administration (e.g., a dose, dosage form, or mode of administration described herein). In another embodiment, the informational material can include instructions to administer a compound described herein to a suitable subject, e.g., a human, e.g., a human having or at risk for a disorder described herein.

The informational material of the kits is not limited in its form. In many cases, the informational material, e.g., instructions, is provided in printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, email address, website, or telephone number, where a user of the kit can obtain substantive information about a compound described herein and/or its use in the methods described herein. Of course, the informational material can also be provided in any combination of formats.

In addition to a compound described herein, the composition of the kit can include other ingredients, such as a solvent or buffer, a stabilizer, a preservative, a flavoring agent (e.g., a bitter antagonist or a sweetener), a fragrance, a dye or coloring agent, for example, to tint or color one or more components in the kit, or other cosmetic ingredient, and/or a second agent for treating a condition or disorder described herein. Alternatively, the other ingredients can be included in the kit, but in different compositions or containers than a compound described herein. In such embodiments, the kit can include instructions for admixing a compound described herein and the other ingredients, or for using a compound described herein together with the other ingredients.

In some embodiments, the components of the kit are stored under inert conditions (e.g., under Nitrogen or another inert gas such as Argon). In some embodiments, the components of the kit are stored under anhydrous conditions (e.g., with a desiccant). In some embodiments, the components are stored in a light blocking container such as an amber vial.

A compound described herein can be provided in any form, e.g., liquid, dried or lyophilized form. It is preferred that a compound described herein be substantially pure and/or sterile. When a compound described herein is provided in a liquid solution, the liquid solution preferably is an aqueous solution, with a sterile aqueous solution being preferred. When a compound described herein is provided as a dried form, reconstitution generally is by the addition of a suitable solvent. The solvent, e.g., sterile water or buffer, can optionally be provided in the kit.

The kit can include one or more containers for the composition containing a compound described herein. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of a compound described herein. For example, the kit includes a plurality of syringes, ampules, foil packets, or blister packs, each containing a single unit dose of a compound described herein. The containers of the kits can be air tight, waterproof (e.g., impermeable to changes in moisture or evaporation), and/or light-tight.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device. In a preferred embodiment, the device is a medical implant device, e.g., packaged for surgical insertion.

### EXAMPLES

### Example 1

### In Vitro Receptor Binding and Functional Assay Results for F-Morphine

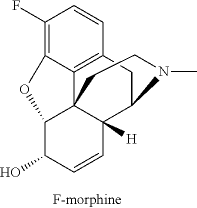

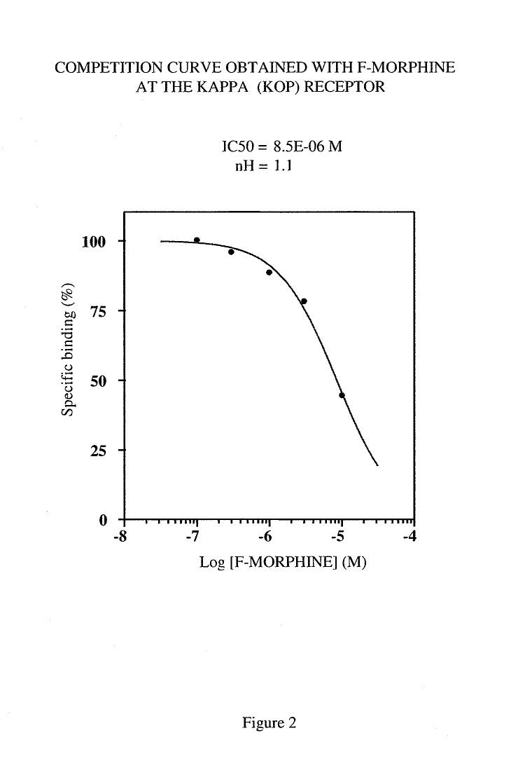

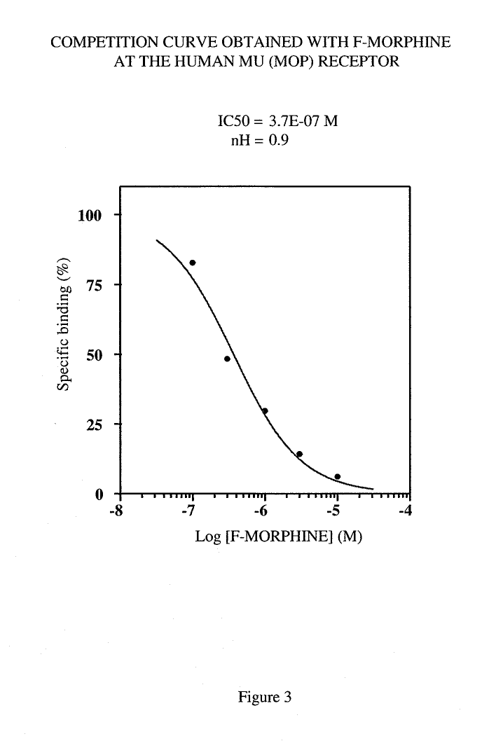

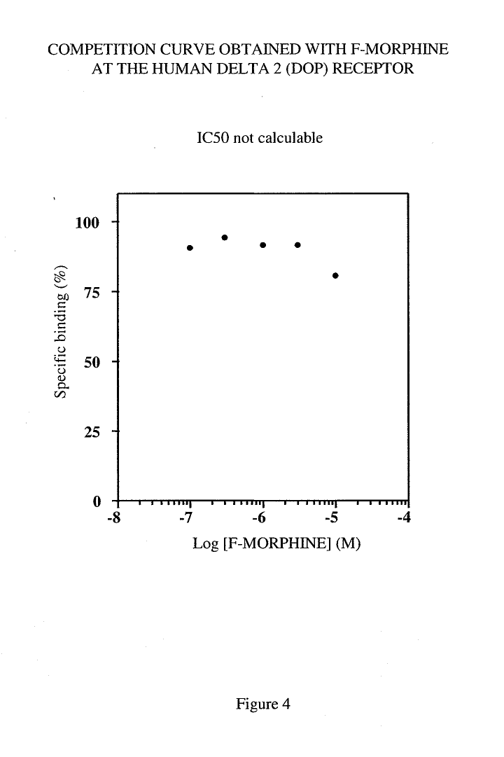

Receptor binding assays (Table 1 and FIGS. 1-4)

More potent at mu than kappa, no observed binding to delta receptor

a. Mu: IC₅₀ 370 nM (Ki 150 nM)

b. Kappa: IC₅₀ 8.5 uM (Ki 5.6 uM)

c. Delta: no significant binding up to 10 uM

Cellular Functional Assays

Functions as an agonist at mu and kappa opioid receptors; EC₅₀ values were not determined.

### Example 2

### In Vivo Pharmacokinetics and Brain Partition Study

Study Design:

single 1 mg/kg iv dose administered in rats under fasted conditions

plasma concentrations determined at 10 time points (2 mins -24 hrs)

brain partition ratio determined at 1 and 4 hours

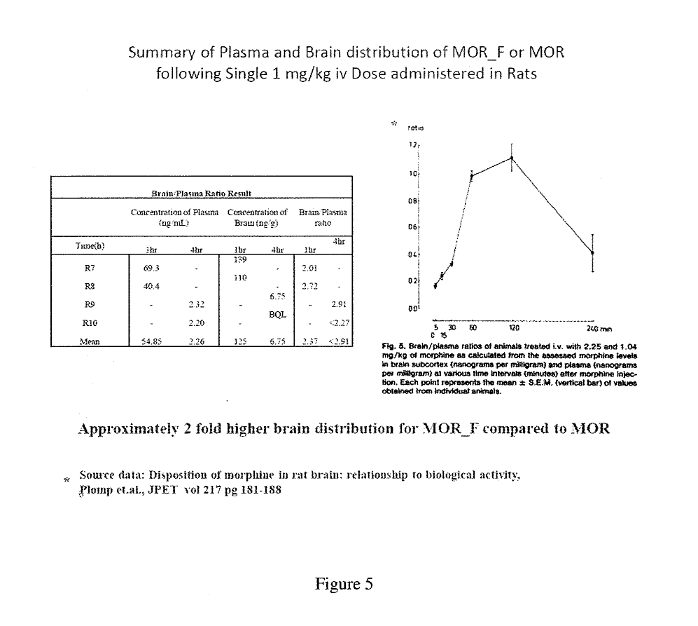

Results (FIGS. 5-7)

fluorination of morphine affects its biodistribution (FIG. 5)
F-morphine partitions well into the brain
   brain/plasma ratios of F-morphine at 1 and 4 hours are between 2 and 3, compared with 1 or less for morphine

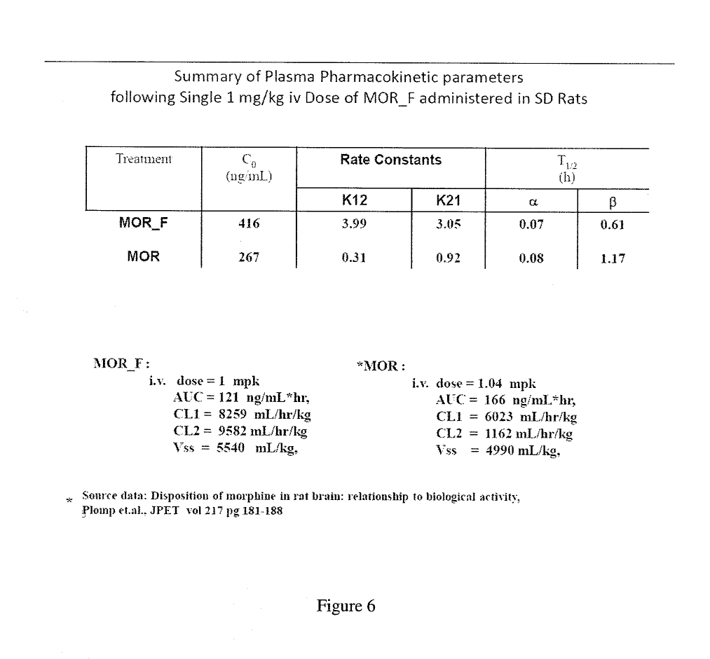

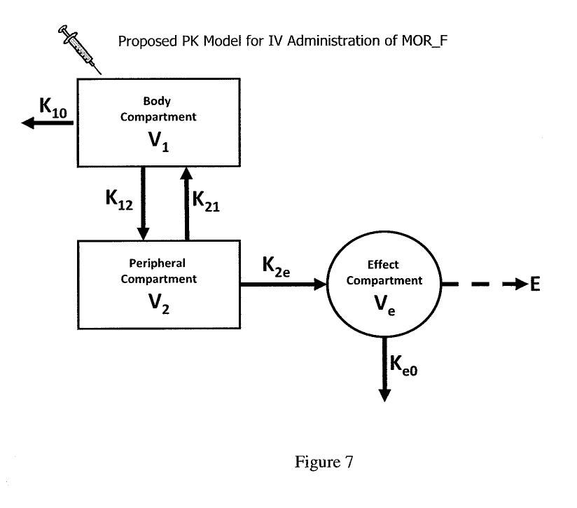

K12/K21 ratio of morphine vs. F-morphine suggests differential effects on potential efflux transporters (data in FIG. 6, K12/K21 model in FIG. 7).

TABLE 1
Summary of In Vitro IC₅₀ Determinations

| | | | | | |
|---|---|---|---|---|---|
| Assay | | | | | |
| Cerep Compound I.D. | Compound | IC₅₀ (M) | Kᵢ (M) | n_{H} | Flags |

opioid (non-selective) (antagonist radioligand)

| | | | | |
|---|---|---|---|---|
| 16350-1 | F-morphine | 8.0E−07 | 5.8E−07 | 0.6 |

δ₂ (DOP) (h) (agonist radioligand)

| | | |
|---|---|---|
| 16350-1 | F-morphine | N.C. |

κ (KOP) (agonist radioligand)

| | | | | |
|---|---|---|---|---|
| 16350-1 | F-morphine | 8.5E−06 | 5.6E−06 | 1.1 |

μ (MOP) (h) (agonist radioligand)

| | | | | |
|---|---|---|---|---|
| 16350-1 | F-morphine | 3.7E−07 | 1.5E−07 | 0.9 |

N.C. IC50 value not calculable. Concentration-response curve shows less than 25% effect at the highest tested concentration.

What is claimed is:

1. A fluorinated morphine.

2. The fluorinated morphine of claim 1, wherein an aryl group has been substituted with one or more fluorine atoms.

3. The fluorinated morphine of claim 2, wherein a hydrogen or a hydroxy substituent of an aryl group has been replaced with a fluorine.

4. The fluorinated morphine of claim 1, wherein the fluorinated morphine has the following formula: 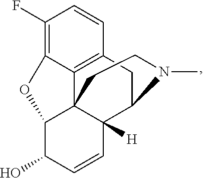
or a pharmaceutically acceptable salt thereof.

5. A fluorinated morphine-6-glucuronide.

6. The fluorinated morphine-6-glucuronide of claim 5, wherein an aryl group has been substituted with one or more fluorine atoms.

7. The fluorinated morphine-6-glucuronide of claim 6, wherein a hydrogen or hydroxy substituent of an aryl group has been replaced with a fluorine.

8. The fluorinated morphine-6-glucuronide of claim 4, wherein the fluorinated morphine-6-glucuronide has the following formula: 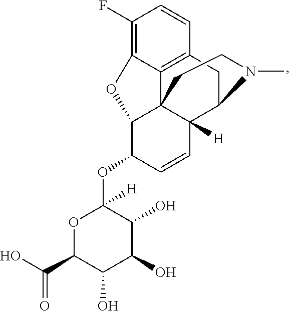
or a pharmaceutically acceptable salt thereof.

9. A fluorinated oxycodone.

10. A fluorinated buprenorphine.

11. A fluorinated naloxone.

12. A fluorinated hydrocodone.

13. A fluorinated dextropropoxyphene.

14. A fluorinated methadone.

15. A fluorinated hydromorphone.

16. A fluorinated codeine.

17. A fluorinated dextromoramide.

18. A fluorinated diamorphine.

19. A fluorinated dihydrocodeine.

20. A fluorinated dipipanone.

21. A fluorinated meptazinol.

22. A fluorinated nalbuphine.

23. A fluorinated lofexidine.

24. A fluorinated naltrexone.

25. A fluorinated oxymorphone.

26. A fluorinated nalorphine.

27. A fluorinated etorphine.

28. A fluorinated dihydroetorphine.

29. A fluorinated N-phenethyl-14-ethoxymetopon.

30. A fluorinated thebaine.

31. An ¹⁸F-substituted clonidine.

32. An ¹⁸F-substituted pentazocine.

33. An ¹⁸F-substituted pethidine.

34. A fluorinated phenazocine of one of the following formulae:

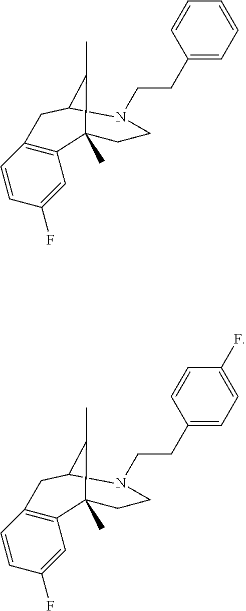

35. An ¹⁸F-substituted phenazocine.
